(19) United States
(12) Patent Application Publication
(10) Pub. No.: US 2023/0162851 A1
(43) Pub. Date: May 25, 2023

(54) METHODS AND SYSTEMS FOR ENHANCING CLINICAL SAFETY OF PSYCHOACTIVE THERAPIES

(71) Applicant: Eleusis Health Solutions US, Inc., New York, NY (US)

(72) Inventors: Neiloufar FAMILY, Brooklyn, NY (US); Shlomi RAZ, Brooklyn, NY (US)

(21) Appl. No.: 17/910,452

(22) PCT Filed: Mar. 9, 2021
(86) PCT No.: PCT/US2021/021474

§ 371 Date: Sep. 9, 2022

Related U.S. Application Data
(60) Provisional application No. 62/987,027, filed on Mar. 9, 2020.

(51) Int. Cl. G16H 40/63; A61B 5/00; A61B 5/11; G16H 20/70

(52) U.S. Cl. CPC G16H 40/63; A61B 5/4848; A61B 5/1113; A61B 5/746; G16H 20/70

(57) ABSTRACT

The present invention provides methods and systems for enhancing clinical safety of psychoactive therapies (e.g., 5-HT2A agonists (e.g., LSD and psilocybin), dissociatives, and empathogens) when administered in a clinical treatment setting. In particular, the invention provides methods and systems for monitoring a patient and an attendant, e.g., via an access module capable of relaying session data and/or deriving a patient response metric.

### BACKGROUND OF THE INVENTION

Psychoactive compounds (e.g., psychedelic, dissociative, and empathogenic compounds) have demonstrated therapeutic effect in a wide variety of clinical applications, including depression, anxiety, PTSD, substance abuse, and a variety of other conditions. The therapeutic utility of psychedelic compounds provides the impetus for the development of supportive technologies to enhance the safety, efficacy, and efficiency of the treatment of conditions with these compounds.

However, therapeutic applications of psychedelic, dissociative, and empathogenic compounds are associated with risks of adverse effects, requiring close clinical monitoring and availability of trained staff, such as physicians. Another significant clinical risk associated with psychoactive therapy is acute vulnerability of the patient to clinical malpractice. Given the profound alteration in perception, inhibition, and suggestibility, patients are acutely vulnerable to clinical malpractice in the context of psychoactive drug therapies.

Thus, in order to enable broad implementation of safe psychoactive drug therapies in the context of public health, there is a need for methods and systems that enable efficient monitoring and scalability of psychoactive therapy programs, provide a centralized platform for training clinical staff (e.g., attendants) responsible for monitoring psychoactive therapy sessions, and enhance patient safety through oversight of clinical practice.

### SUMMARY OF THE INVENTION

In one aspect, provided herein is a method of monitoring a psychoactive therapy session. The method includes: (i) receiving session data via an access module (e.g., uploading session data onto an access module); and (ii) transferring the session data from the access module to a remote monitor (e.g., in real time, e.g., as a live video and/or audio stream). The session data is transmitted to the access module from the psychoactive therapy session, wherein the psychoactive therapy session is conducted in a treatment setting including a patient and an attendant (e.g., an attendant without specialized clinical certification), wherein no physician (e.g., no psychiatrist) is present in the treatment setting. In some embodiments, the session data is processed within the access module (e.g., within a processor) to derive a patient response metric. In some embodiments, the method further includes transferring the patient response metric from the access module to the remote monitor (e.g., informing the remote monitor of the patient response metric).

In another aspect, provided herein is a method of monitoring a psychoactive therapy session, the method comprising: (i) receiving session data via an access module (e.g., uploading session data onto an access module), and (ii) within the access module, processing the session data to derive a patient response metric indicative of a patient response to the psychoactive therapy session. The session data is transmitted to the access module (e.g., within a processor) from the psychoactive therapy session. The psychoactive therapy session is conducted in a treatment setting comprising a patient and an attendant, wherein no physician (e.g., no psychiatrist) is present in the treatment setting. In some embodiments, the method further comprises transferring the patient response metric from the access module to a remote monitor (e.g., informing the remote monitor of the patient response metric) and/or transferring unprocessed session data from the access module to the remote monitor.

In some embodiments of any of the preceding methods, the session data is transferred from the access module to the remote monitor in real-time (e.g., as a live video and/or audio stream). For example, in some embodiments, the session data comprises digitally recorded data (e.g., video and/or audio data). Additionally or alternatively, the digitally recorded data comprises wearable sensor data (e.g., including acceleration data, movement data, or biometric data (e.g., heart rate, breathing characteristics, body temperature, electroencephalogram (EEG) data, etc.).

In some embodiments, the digitally recorded data comprises infrared sensor data. In some embodiments, the patient response metric comprises a measure of movement throughout the treatment setting derived from the digitally recorded data. In some embodiments, a high measure of movement throughout the treatment setting indicates an adverse patient response to the psychoactive therapy session.

In some embodiments, the patient response metric comprises a measure of conversation between the patient and the attendant. In some embodiments, a lengthy inaudible conversation between the patient and the attendant indicates a deviation from protocol (and may trigger summoning clinical support to the treatment setting, e.g., thereby intervening in the psychoactive therapy session). In some embodiments, the length inaudible conversation is at least 10 seconds, at least 15 seconds, at least 20 seconds, at least 30 seconds, at least 45 seconds, at least 60 seconds, at least two minutes, at least three minutes, at least four minutes, at least five minutes, at least six minutes, at least seven minutes, at least eight minutes, at least nine minutes, or at least 10 minutes long (e.g., between 10 seconds and 10 minutes, between 15 seconds and 10 minutes, between 30 seconds and 10 minutes, between 45 seconds and 10 minutes, between 60 seconds and 10 minutes, between two minutes and 10 minutes, between three minutes and 10 minutes, between four minutes and 10 minutes, or between five minutes and ten minutes long).

In some embodiments, the method further comprises alerting clinical support of the adverse patient response. In some embodiments, the clinical support includes a physician (e.g., a psychiatrist). Additionally or alternatively, the clinical support can include one or more non-physician clinical professionals (e.g., non-physician clinical professionals specifically trained to address adverse patient response). Non-physician clinical professionals include clinical practitioners, such as certified mental health practitioners. In some embodiments, the clinical support staff member is the remote monitor, a non-certified staff member, a certified, clinician, or a physician. In some embodiments, after alerting the clinical support staff member of the adverse patient response, the clinical support staff member: (a) intervenes in the psychoactive therapy session; (b) alerts another clinical support staff member; and/or (c) escalates the alert to a physician. In some embodiments, part (b) further comprises escalating the alert to a physician. In some embodiments, the physician intervenes in the psychoactive therapy session. In some embodiments, the intervention is a remote intervention (e.g., by remotely requesting local clinical support) or a local intervention (e.g., by physically entering the treatment facility).

In some embodiments of any of the preceding aspects, the session data comprises manually recorded data provided by the attendant (e.g., a rating of unresponsiveness). Additionally or alternatively, the manually recorded data comprises a patient self-report (e.g., through a questionnaire, e.g., a questionnaire administered by the attendant).

In some embodiments, the patient response metric comprises a measure of anxiety and/or paranoid ideation (e.g., derived from manually recorded data or digitally recorded data). In some embodiments, a high measure of anxiety and/or paranoid ideation indicates an adverse patient response to the psychoactive therapy session. In some embodiments, the method further includes alerting clinical support of the adverse patient response.

In another aspect, provided herein is a method of intervening in a psychoactive therapy session. The method includes: (i) receiving session data via an access module (e.g., uploading session data onto an access module), wherein the session data is transmitted to the access module from the psychoactive therapy session, wherein the psychoactive therapy session is conducted in a treatment setting comprising a patient and an attendant, and wherein no physician (e.g., psychiatrist) is present in the treatment setting; (ii) transferring the session data from the access module to a remote monitor; (iii) identifying an aberrance in the session data; and (iv) summoning clinical support to the treatment setting, thereby intervening in the psychoactive therapy session. In some embodiments, the aberrance is a deviation from protocol and/or misconduct by the attendant. In some embodiments, the method further includes processing the session data (e.g., within the access module, e.g., within a processor) to derive a patient response metric. In some embodiments, the method further includes transferring the patient response metric from the access module to the remote monitor. In some embodiments, the aberrance is an adverse patient response to the psychoactive therapy session. In some embodiments, the aberrance is deviation from protocol.

In some embodiments of any of the previous methods, the access module and/or remote monitor receives session data from multiple treatment settings (e.g., the session data is received simultaneously). In some embodiments, the multiple treatment settings include from 2-1,000 treatment settings, 3-500 treatment settings, 4-200 treatment settings, 5-100 treatment settings, 6-50 treatment settings, 7-40 treatment settings, 8-30 treatment settings, 9-25 treatment settings, or 10-20 treatment settings (e.g., from 2-5 treatment settings, 5-10 treatment settings, 10-20 treatment settings, 20-30 treatment settings, 30-40 treatment settings, 40-50 treatment settings, 50-100 treatment settings, 100-200 treatment settings, or more than 200 treatment settings, e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, or more treatment settings).

In some embodiments of any of the previous methods, the attendant is equipped with a line of communication to the remote monitor (e.g., a secure line of communication) for direct communication (e.g., to summon the monitor to the treatment setting to respond to a stimulus not derived from session data). For example, a remote monitor may be summoned by the attendant to respond for technical support or temporary coverage, e.g., for bathroom breaks. In some embodiments, the line of communication between the attendant and monitor is a two-way line of communication (e.g., a secure two-way line of communication).

In some embodiments of any of the previous methods, the remote monitor is not a physician (e.g., not a psychiatrist). In some embodiments, the remote monitor is equipped with a secure connection to a physician.

In another aspect, the invention is method of intervening in a psychoactive therapy session, the method comprising: (i) receiving session data via an access module (e.g., uploading session data onto an access module), wherein the session data is transmitted to the access module from the psychoactive therapy session, wherein the psychoactive therapy session is conducted in a treatment setting comprising a patient and an attendant, and wherein no physician (e.g., no psychiatrist) is present in the treatment setting; (ii) within the access module (e.g., within a processor), processing the session data to derive a patient response metric indicative of a patient response to the psychoactive therapy session; (iii) identifying an aberrance based on the patient response metric; and (iv) summoning clinical support to the module to the remote monitor. In some embodiments, the session data comprises digitally recorded data, such as video and/or audio data. Additionally or alternatively, the digitally recorded data comprises wearable sensor data (e.g., including acceleration data, movement data, or biometric data (e.g., heart rate, breathing characteristics, body temperature, EEG data, etc.).

In some embodiments, the digitally recorded data comprises infrared sensor data. In some embodiments, the patient response metric comprises a measure of movement throughout the treatment setting derived from the digitally recorded data. In some embodiments, a high measure of movement throughout the treatment setting indicates an adverse patient response to the psychoactive therapy session.

In some embodiments, the patient response metric comprises a measure of conversation between the patient and the attendant. In some embodiments, a lengthy inaudible conversation between the patient and the attendant indicates a deviation from protocol. In some embodiments, the length inaudible conversation is at least 10 seconds, at least 15 seconds, at least 20 seconds, at least 30 seconds, at least 45 seconds, at least 60 seconds, at least two minutes, at least three minutes, at least four minutes, at least five minutes, at least six minutes, at least seven minutes, at least eight minutes, at least nine minutes, or at least 10 minutes long (e.g., between 10 seconds and 10 minutes, between 15 seconds and 10 minutes, between 30 seconds and 10 minutes, between 45 seconds and 10 minutes, between 60 seconds and 10 minutes, between two minutes and 10 minutes, between three minutes and 10 minutes, between four minutes and 10 minutes, or between five minutes and ten minutes long).

In some embodiments of any of the preceding aspects, the session data comprises manually recorded data provided by the attendant (e.g., a rating of unresponsiveness). Additionally or alternatively, the manually recorded data comprises a patient self-report.

In some embodiments, the patient response metric comprises a measure of anxiety and/or paranoid ideation (e.g., derived from manually recorded data or digitally recorded data). In some embodiments, a high measure of anxiety and/or paranoid ideation indicates an adverse patient response to the psychoactive therapy session. In some embodiments, the method further includes alerting clinical support of the adverse patient response.

In another aspect, provided herein is a method of training clinical staff for monitoring a psychoactive therapy session, the method comprising: (i) performing a method of any one of the preceding methods; (ii) providing one or more trainees; (iii) presenting session data to the one or more trainees; and (iv) providing a characterization of the session data to the one or more trainees, thereby training clinical staff. In some embodiments, the clinical staff is an attendant, a remote monitor, a clinical professional, or a physician (e.g., a psychiatrist).

In another aspect, provided is a method of conducting a psychoactive therapy session, the method comprising: (i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; (ii) within the access module, processing the session data to derive a first patient response metric indicative of a psychoactive drug effect, and processing the session data to derive a subsequent patient response metric indicative of the psychoactive drug effect; (iii) comparing the first patient response metric with the subsequent patient response metric; and (iv) based on the comparison of (iii), determining whether to dismiss the patient from the treatment setting. In some embodiments, the method further includes dismissing the patient from the treatment setting. In some embodiments, the access module comprises a processor, and wherein the processor performs the comparison.

In another aspect, the invention includes a method of editing session data from a psychoactive therapy session, the method comprising: (i) receiving session data via an access module, wherein the session data is transmitted to the access module from the psychoactive therapy session; and (ii) within the access module: (a) processing the session data comprising a pertinent dataset and an extraneous dataset to derive a patient response metric indicative of a patient response to the psychoactive therapy session, wherein the patient response metric is preferentially based on the pertinent dataset; and (b) producing an edited dataset by isolating the pertinent dataset from the extraneous dataset.

In another aspect, the invention includes a method of generating a patient response database, the method comprising: (i) via an access module, receiving session data from a first psychoactive therapy session and session data from a second psychoactive therapy session, wherein the first and second psychoactive therapy sessions are each conducted in a treatment setting comprising a patient and an attendant, wherein no physician is present in the treatment setting during either of the first and second psychoactive therapy sessions; (ii) within the access module: (a) processing the session data from the first psychoactive therapy session to derive a first patient response metric indicative of a first patient response to the psychoactive therapy session; and (b) processing the session data from the second psychoactive therapy session to derive a second patient response metric indicative of a second patient response to the psychoactive therapy session; and (iii) recording the first patient response metric and the second patient response metric to a computer-readable medium, thereby generating a patient response database. In some embodiments, the method further includes deriving an average patient response from the first patient response and the second patient response. In some embodiments, the invention further includes receiving session data from a third, fourth, fifth, sixth, and/or seventh psychoactive therapy session each conducted in a treatment setting comprising a patient and an attendant, wherein no physician is present in the treatment setting during any of the psychoactive therapy sessions; within the access module: processing the session data from the third, fourth, fifth, sixth, and/or seventh psychoactive therapy sessions to derive a third, fourth, fifth, sixth, and/or seventh patient response metric indicative of a third, fourth, fifth, sixth, and/or seventh patient response to the psychoactive therapy session; and (b) integrating the session data from the third, fourth, fifth, sixth, and/or seventh psychoactive therapy sessions with the session data from the first and second psychoactive therapy sessions to derive a third, fourth, fifth, sixth, and/or seventh patient response metric indicative of a third, fourth, fifth, sixth, and/or seventh patient response to the psychoactive therapy session; and (iii) recording the third, fourth, fifth, sixth, and/or seventh patient response metric to a computer-readable medium.

In another aspect, the invention provides a method of monitoring a psychoactive therapy session, the method comprising: (i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; and (ii) within the access module, processing the session data to derive a patient response metric indicative of a patient response to the psychoactive therapy session, wherein the processing draws reference data from a patient response database generated as disclosed herein (e.g., by a method of generating a patient response database, the method comprising: (i) via an access module, receiving session data from a first psychoactive therapy session and session data from a second psychoactive therapy session, wherein the first and second psychoactive therapy sessions are each conducted in a treatment setting comprising a patient and an attendant, wherein no physician is present in the treatment setting during either of the first and second psychoactive therapy sessions; (ii) within the access module: (a) processing the session data from the first psychoactive therapy session to derive a first patient response metric indicative of a first patient response to the psychoactive therapy session; and (b) processing the session data from the second psychoactive therapy session to derive a second patient response metric indicative of a second patient response to the psychoactive therapy session; and (iii) recording the first patient response metric and the second patient response metric to a computer-readable medium, thereby generating a patient response database (e.g., wherein the method further includes deriving an average patient response from the first patient response and the second patient response)).

In some embodiments of any of the preceding methods, the psychoactive therapy session is for treatment of a psychological condition (e.g., depression, anxiety, PTSD, substance abuse, alcoholism, anorexia nervosa, bulimia, obesity, eating disorders, or obsessive-compulsive disorder). In some embodiments of any of the preceding methods, the psychoactive therapy session is for treatment of a non-psychological condition (e.g., an autoimmune disorder, or pain (e.g., pain syndromes, migraine, cluster headaches, neuropathic pain, fibromyalgia, etc.)).

In some embodiments of any of the preceding methods, the psychoactive therapy session is part of a neurorehabilitation program. In some embodiments, the psychoactive therapy session is for enhancing well-being.

In some embodiments of any of the preceding methods, the psychoactive therapy session is conducted in the presence of one and only one attendant (e.g., a single attendant, e.g., the patient and the attendant are the only people in the treatment setting during the psychoactive therapy session).

In some embodiments of any of the preceding methods, the psychedelic therapy session comprises a perceptual dose of a psychoactive agent (e.g., a dose of greater than about 0.1 μg/kg, greater than about 0.5 μg/kg, greater than about 1.0 μg/kg, greater than about 5.0 μg/kg, greater than about 10 μg/kg, greater than about 20 μg/kg, greater than about 50 μg/kg, greater than about 100 μg/kg, greater than about 200 μg/kg, greater than about 500 μg/kg, greater than about 1.0 mg/kg, greater than about 5.0 mg/kg, greater than about 10 mg/kg, greater than about 50 mg/kg, or greater than about 100 mg/kg body weight, e.g., from about 0.1 μg/kg to about 0.5 μg/kg, from about 0.5 μg/kg to about 10 μg/kg, from about 1.0 μg/kg to about 5.0 μg/kg, from about 5.0 μg/kg to about 10 μg/kg, from about 10 μg/kg to about 50 μg/kg, from about 50 μg/kg to about 100 μg/kg, from about 100 μg/kg to about 500 μg/kg, from about 500 μg/kg to about 1.0 mg/kg, from about 1.0 mg/kg to about 10 mg/kg, from about 10 mg/kg to about 50 mg/kg, from about 50 mg/kg to about 100 mg/kg, or from about 100 mg/kg to about 500 mg/kg).

In some embodiments of any of the preceding methods, the psychoactive therapy session comprises administration of an agent selected from the group consisting of a 5-HT2A receptor agonist, an empathogenic agent, and a dissociative agent. In some embodiments, the psychoactive therapy session comprises administration of a 5-HT2A receptor agonist. In some embodiments, the 5-HT2A receptor agonist is selected from lysergic acid diethylamide (LSD), psilocybin, DOI (±)-1-(2,5-dimethoxyphenyl)-2-aminopropane hydrochloride; (R)-DOI ((R)-1-(2,5-dimethoxy-4-iodophenyl)-2-aminopropane); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl)methylamine) ayahuasca; 3,4,5-trimethoxyphenethylamine (mescaline); 5-methoxy-N,N-dimethyltryptamine (5-meo-DMT); ibogaine; voacangine; noribogaine; coronaridine; 18-methoxycoronaridine (18-mc); methoxyethyl 18-MC; 18-methylaminocoronaridine (18-MAC); 2-methoxyethyl-18-methoxycoronaridinate; a compound of

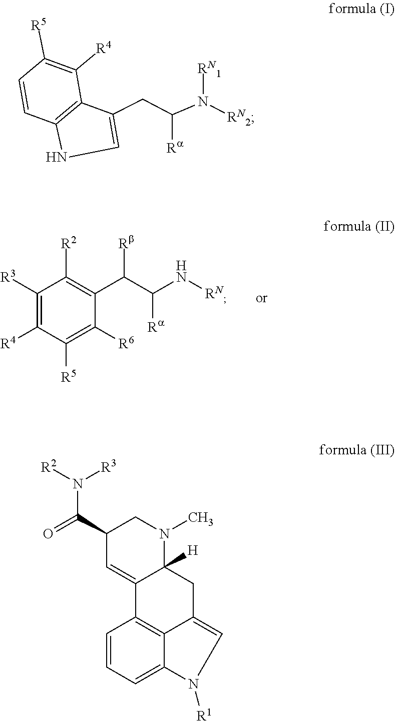

or a pharmaceutically acceptable salt thereof. In some embodiments, the 5-HT_{2A} receptor agonist is LSD. In some embodiments, the LSD is administered at a dose from 1-500 μg (e.g., 50-100 μg, e.g., about 50 μg, about 60 μg, about 70 μg, about 80 μg, about 90 μg, or about 100 μg).

In some embodiments, the psychoactive therapy session comprises administration of an empathogenic agent, such as 3,4-methylenedioxymethamphetamine (MDMA).

In some embodiments, the psychoactive therapy session comprises administration of a dissociative agent, such as NMDA antagonists (e.g., ketamine, esketamine, or arketamine).

In some embodiments of any of the above methods, the session data includes wearable sensor data. In particular embodiments, the sensor data includes EEG sensor data or infrared sensor data.

In some aspects, the invention includes a system (e.g., a system comprising an access module described herein), wherein the system is configured to perform the method of any of the preceding aspects.

As used herein, “session data” refers to information obtained from a patient during a psychoactive therapy session. Session data includes raw data (e.g., unprocessed video or audio recordings, attendant notes, or other entries of information during the session) and processed data (e.g., processed video or audio recordings, metrics derived (directly or indirectly) from raw session data (e.g., a patient response metric). In some embodiments, the session data is raw data. In other embodiments, the session data is processed data.

As used herein, an “access module” refers to any hardware and/or software (or system thereof) that receives session data (e.g., raw session data and/or processed session data) and (i) processes the session data; and/or (ii) relays the session data to a remote monitor. In some embodiments, the access module receives the session data (e.g., raw session data) and processes the session data (e.g., to derive a patient response metric available to a remote monitor, physician, or clinical support staff). In some embodiments, the access module receives the session data (e.g., raw session data) and relays the session data to a remote monitor (e.g., via real-time stream). In some embodiments, the access module receives the session data (e.g., raw session data), processes the session data (e.g., to derive a patient response metric), and relays the processed session data to a remote monitor, physician, or clinical support staff.

As used herein, a “treatment setting” is a physical space (e.g., a room or a suite) which is regulated by clinical standards, e.g., for safety and/or data control.

As used herein, a “physician” is a person who has a Doctor of Medicine degree (M.D.; such as a psychiatrist or psychotherapist) or Osteopathic Medicine degree (D.O.) who is legally authorized by local government to practice medicine, such as a person who has a Ph.D. in clinical psychology (i.e., a clinical psychologist).

As used herein, a “clinical practitioner” is a nurse practitioner, clinical social worker, or physician assistant who is authorized by state or local government to practice within the scope of their practice as defined under state or local law. In some embodiments, a clinical practitioner is certified to address an adverse effect associated with administration of a psychotherapy.

As used herein, a “certified mental health practitioner” is a person authorized by state or local government to practice in the field of mental health, such as a mental health nurse. In some embodiments, a certified mental health practitioner is certified to address an adverse effect associated with administration of a psychotherapy.

As used herein, an “attendant” is a person who is not a physician and who is physically present in the same room as the patient for at least a portion of the psychedelic therapy session. In some embodiments, the attendant may not be certified as a mental health practitioner, but has been qualified to be an attendant via participation in a training program for attendants, passing a certification exam, and/or participating in ongoing training (e.g., according to method or system of training as provided herein).

As used herein, a “remote monitor” is a person who is not physically present in the same room as the patient for at least a portion of the psychedelic therapy session and who has access to a recording of the psychedelic therapy session. In some instances, the remote monitor is not a physician. In other instances, the remote monitor is a physician. In some instances, the remote monitor is not a clinical practitioner. In other instances, the remote monitor is a clinical practitioner. In some instances, the remote monitor is certified, e.g., in clinical research, clinical trial management, etc.

As used herein, to “derive” a metric from a recording refers to the act of obtaining the metric using information provided by the recording, alone or in combination with additional information not provided by the recording (e.g., using a classifier or, alternatively, by comparing session data from a given psychoactive therapy session to session data from a previous psychoactive therapy session). For example, a patient response metric may be derived from a video recording by processing all or a portion of the video recording to obtain a measure of motor activity, and, if the measure of motor activity exceeds a predetermined threshold value by a factor of X, a patient response metric having a value of Y is derived. In such cases, the predetermined threshold may be set using a classifier (e.g., using a cross-validation approach with training data). One or more data sets from a recording may be input into an algorithm (e.g., an algorithm having preset and/or variable factors, e.g., a machine learning algorithm (e.g., a Random Forest or Support Vector Machine), used in accordance with methods known in the art and described herein), the product of which is a metric derived from the recording. In another example, session data from one or more psychoactive therapy sessions is compared to session data from one or more previous psychoactive therapy sessions (e.g., among the same patient).

As used herein, a “patient response metric” is a measure of the patient's response to the psychedelic therapy being administered, which can be derived from one or more parameters of session data. The response can be a therapy-induced altered state of consciousness, distress, anxiety, paranoia, dread, and/or other psychoactive drug effects (e.g., acute psychoactive drug effects). In some embodiments, a patient response metric discriminates between a psychopathology (e.g., bipolar disorder (e.g., bipolar mania) or schizophrenia) and a positive or adverse drug effect and serves as a predictor of treatment response. Patient response metrics include locomotion, unresponsiveness to a question, other language or behavioral characteristics, or a combination thereof. In some instances, the patient response metric is derived from multiple parameters, wherein the multiple parameters are obtained through one or more data streams (e.g., digitally recorded data (e.g., audio, video, and/or biometric data) and/or manually recorded data (e.g., data recorded by the attendant)).

As used herein, an “aberrance” is information (e.g., session data) associated with a negative event, such as an adverse patient response or deviation from protocol during the session, e.g., misconduct by the attendant. In embodiments of the invention in which the aberrance is a deviation from protocol, the protocol (and deviation thereof) may be based on a predetermined risk management plan (e.g., a European Medicines Agency (EMA) Risk Management Plan and/or an FDA Risk Evaluation and Mitigation Strategy (REMS)).

As used herein, a “psychological disorder” refers to a condition characterized by a disturbance in one's emotional or behavioral regulation that reflects a dysfunction in the psychological, biological, or developmental processes underlying mental function. Psychological disorders include, but are not limited to depressive disorders (major depression, melancholic depression, atypical depression, or dysthymia), anxiety disorders (end of life anxiety, generalized anxiety disorder, panic disorder, social anxiety, post-traumatic stress disorder, acute stress disorder, obsessive compulsive disorder, or social phobia), addictions (e.g., substance abuse, e.g., alcoholism, tobacco abuse, or drug abuse)), and compulsive behavior disorders (e.g., primary impulse-control disorders or obsessive-compulsive disorder). Psychological disorders can be any psychological condition associated with one or more symptoms, e.g., somatic symptoms (e.g., chronic pain, anxiety disproportionate to severity of physical complaints, pain disorder, body dysmorphia, conversion (i.e., loss of bodily function due to anxiety), hysteria, or neurological conditions without identifiable cause), or psychosomatic symptoms. Psychological disorders also include repetitive body-focused behaviors, such as tic disorders (e.g., Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder). In some cases, development of a psychological disorder is associated with or characterized by a prodromal symptom, such as depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, and decreased initiation of voluntary responses.

Diagnostic guidance for psychological disorders can be found, for example, in the ICD-10 (The ICD-10 Classification of Mental and Behavioral Disorders: Diagnostic Criteria for Research, Geneva: World Health Organization, 1993) and the DSM-V (American Psychiatric Association. Diagnostic and Statistical Manual of Mental Disorders, Fifth Edition (DSM-V) Arlington, Va.; American Psychiatric Association, 2013).

As used herein, “remote intervention” refers to an intervention that is conducted by a physician (e.g., a psychiatrist) who is not physically present at the treatment setting (i.e., remote) at the time of the intervention. The physician may direct the treatment of a patient from a remote location, e.g., by authorizing treatment to be administered by a clinical practitioner or a certified mental health practitioner who is not a physician. Authorization may be granted from a physician to an attendant, a monitor, and/or another support staff member. In some instances, the physician remotely intervenes after being alerted by an attendant, a remote monitor, or another clinical support staff member. For example, in some embodiments, remote intervention includes authorization by a physician to a non-physician to intervene in the psychoactive therapy session, e.g., by administering a rescue drug, e.g., benzodiazepine.

As used herein, “local intervention” refers to an intervention that is conducted by a physician (e.g., a psychiatrist) who is physically present at the treatment setting at the time of the intervention. In some instances, the physician is summoned to the treatment setting by an attendant, a remote monitor, or another clinical support staff member to locally intervene.

As used herein, “well-being” refers to a positive state of health or comfort, e.g., relative to a reference population. As used herein “mental well-being” refers to a positive mental state, relative to a reference population. For example, in an individual having depression, low self-esteem, addiction, compulsion, or anxiety may experience an improvement in mental well-being in response to therapy aimed at improving mood, self-esteem, addiction, compulsion, or anxiety, respectively. As used herein, “physical well-being” refers to one or more positive aspects of an individual's physical health. For example, an improvement of physical well-being includes alleviation of somatic symptoms associated with a psychological disorder, depression, addiction, compulsion, anxiety, or sexual dysfunction. Such symptoms include, for example, chronic pain, pain disorder, relational disorder, body dysmorphia, conversion (e.g., loss of bodily function due to anxiety), hysteria, neurological conditions without identifiable cause, or psychosomatic illness).

As used herein, a “psychedelic agent” refers to a compound capable of inducing an altered state of consciousness, i.e., a marked deviation in the subjective experience or psychological functioning of a normal individual from his or her usual waking consciousness. Altered states of consciousness can be monitored, evaluated, and/or quantified using any of a variety of methods known in the art including, without limitation, Dittrich's APZ (Abnormal Mental States) questionnaire, and its revised versions, OAV and 5D-ASC (see, for example, Dittrich et al., A Pharmacopsychiatry 1998, 31:80; Studerus et al., PLoS ONE2010, 5). Psychedelic agents include 5-HT_{2A} agonists (e.g., lysergic acid diethylamide (LSD), empathogenic agents (i.e., serotonin (5-HT) releasing agents; e.g., MDMA), and dissociative agents (i.e., N-Methyl-D-aspartate (NMDA) receptor agonists; e.g., ketamine).

As used herein, a “5-HT_{2A} agonist” refers to a compound that increases the activity of a 5-hydroxytryptamine 2A receptor. Examples of such agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl)methylamine; ayahuasca; 3,4,5-trimethoxyphenethylamine (mescaline); 5-methoxy-N,N-dimethyltryptamine (5-meo-DMT); ibogaine; voacangine; noribogaine; coronaridine; 18-methoxycoronaridine (18-mc); methoxyethyl 18-MC; 18-methylaminocoronaridine (18-MAC); 2-methoxyethyl-18-methoxycoronaridinate; a compound of formula (I), wherein R^{α}, R^{N}₁, R^{N}₂, R⁴, and/or R⁵ are selected from the group consisting of C, CH₃, OH, F, OCH₃, and H; a compound of formula (II), wherein R^{α}, RD, R², R³, R⁴, R⁵, R⁶, and/or R^{N} are selected from the group consisting of OCH₃, CH₃, SCH₃, Br, I, CH₂CH(CH₃)₂, and H; or a compound of formula (III), wherein R¹, R², and/or R³ are selected from the group consisting of CH₂CH₃, CH(CH₃)CH₂CH₃, CH(CH₃)CH₂CH₂CH₃, C₂H₅, CH₂CH₂CH₃, CH(CH₃)₂, and H. Compounds of formulas (I)-(III) can be synthesized using methods known in the art, including, for example, procedures described in Kornfeld et al. (J. Am. Chem. Soc. 1954, 76(20):5256-5257), Kornfeld et al. (J. Am. Chem. Soc. 1956, 78(13):3087-3114), Marino et al. (J. Org. Chem. 1995, 60(9):2704-2713), and Tsao (J. Am. Chem. Soc. 1951, 73(11):5495-5496).

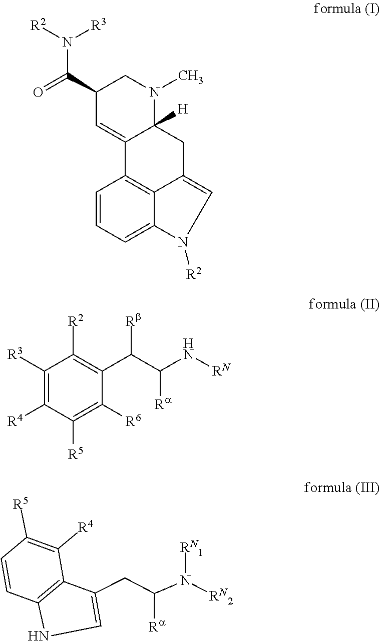

As used herein, the term “treating” refers to administering a pharmaceutical composition for therapeutic purposes. To “treat a disorder” or use for “therapeutic treatment” refers to administering treatment to a patient already suffering from a disease to ameliorate the disease or one or more symptoms thereof to improve the patient's condition. The methods of the invention can also be used as a primary prevention measure, i.e., to prevent a condition or to reduce the risk of developing a condition. Prevention refers to prophylactic treatment of a patient who may not have fully developed a condition or disorder, but who is susceptible to, or otherwise at risk of, the condition. Thus, in the claims and embodiments, the methods of the invention can be used either for therapeutic or prophylactic purposes.

The term “administration” or “administering” refers to a method of giving a dosage of a pharmaceutical composition to a subject, where the method is, e.g., oral, topical, transdermal, by inhalation, intravenous, intraperitoneal, intracerebroventricular, intrathecal, or intramuscular.

As used herein, a “psychotherapy” refers to a non-pharmaceutical therapy in which the subject is psychologically engaged, directly or indirectly (e.g., by dialogue), in an effort to restore a normal psychological condition; to reduce the risk of developing a psychological condition, disorder, or one or more symptoms thereof; and/or to alleviate a psychological condition, disorder, or one or more symptoms thereof. Psychotherapy includes Behavioral Activation (BA), Cognitive Behavioral Therapy (CBT), Interpersonal psychotherapy (IPT), Psychoanalysis, Hypnotherapy, Psychedelic Psychotherapy, Psycholytic Psychotherapy, and other therapies. In some embodiments, a subject undergoes psychotherapy in conjunction with (e.g., prior to, during, and/or after) a pharmaceutical therapy, such as a psychedelic therapy.

Other features and advantages of the invention will be apparent from the following Detailed Description, Examples, Figure, and Claims.

### BRIEF DESCRIPTION OF THE DRAWINGS

The following drawings are illustrative of particular embodiments of the present invention and are not limited to various embodiments encompassed by the present invention.

FIG. 1 is a flow chart illustrating various exemplary elements of a system or method of monitoring a patient undergoing treatment with a psychoactive agent. The patient and the attendant are in the treatment setting (e.g., a room or a suite), which is being monitored digitally by a video recording device that transmits session data to an access module. The remote monitor views the live feed of the video session data. The digitally recorded data is also input to a processor, which compares the data to reference data to derive a patient response metric, which is communicated to the remote monitor and/or a physician, if necessary. Session data is also manually recorded by the attendant through a questionnaire. The manually recorded session data is input to the processor of the access module, alone or in combination with the digitally recorded data, derive the patient response metric communicated to the remote monitor. Manually recorded data can additionally be transmitted directly to the remote monitor, i.e., without passing through the access module. The remote monitor accesses the session data (as raw data (e.g., a live feed) and/or processed data (e.g., a patient response metric)) and can relay information, as necessary, to a clinical support staff and/or a physician, e.g., through a secure connection.

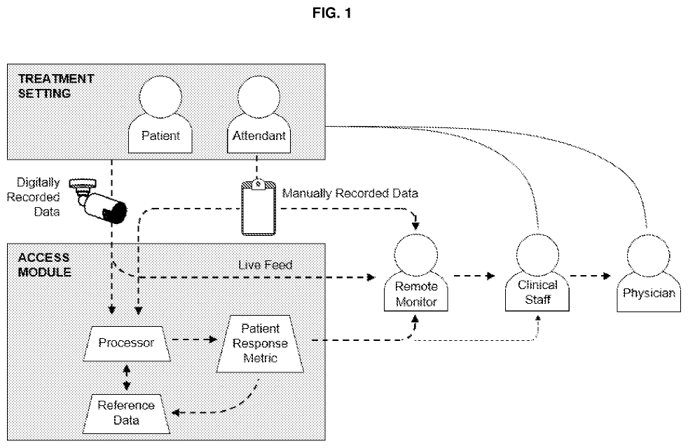

### DETAILED DESCRIPTION

To enable the broad provision of psychoactive drug therapies, methods of providing such therapies with minimal staffing burden is essential, especially given the relative duration of drug effects and required monitoring. The capacity to trigger an escalation for support via device/monitoring system enables for escalation without interruption of clinical supervision of a patient in a situation in which a psychiatrist is not located on premises.

The present invention provides methods for monitoring (e.g., video/audio monitoring) of drug therapy sessions (e.g., psychoactive drug therapy sessions, e.g., in-patient psychoactive drug therapy sessions). Methods of the invention can be used to discriminate between drug states and/or psychopathologies of a patient using session data obtained automatically (e.g., through digital recording (e.g., video and/or audio recording) or manually (e.g., by an attendant or a remote monitor). Methods of the invention include an access module equipped with a processor that can derive information from the session data (e.g., through comparison to reference data, e.g., using known machine learning and/or artificial intelligence (AI) methods). In some instances, the present methods include live-streaming video and audio data to an access module (e.g., a monitoring station) either on-site or off-site, which relays the data to staff (e.g., one or more remote monitors) and/or AI-powered behavioral analytic software programs to detect (e.g., automatically) abnormal behavior in the room, which can indicate a patient's response to the therapy (e.g., an adverse response) and trigger escalation or intervention by clinical staff or psychiatrist. The methods also include using data capture from the patient (e.g., movement, voice, language, questionnaire data, interview data, etc.) to determine whether the patient can be released from care. In addition, the method includes using data capture to train clinicians (e.g., attendants, remote monitors, and physicians). An essential component of the safe and efficacious provision of psychoactive drug therapy is the training of clinicians responsible for providing safety monitoring and adjunctive psychotherapy. Ongoing training of clinicians is an essential aspect of improving clinical care, and scalable and cost-effective methods of facilitating ongoing training are required for the continuous improvement of psychoactive drug therapy service provision.

### Methods and Systems of Monitoring

In some instances, the methods of the invention include monitoring a psychoactive therapy session conducted in a treatment setting, such as a clinical room, suite, or other controlled facility. During the psychoactive therapy session, the patient is accompanied (i.e., physically accompanied in the treatment setting) by an attendant (e.g., an attendant without specialized clinical certification) and not a physician (e.g., no psychiatrist). In some instances, there is one and only one attendant (e.g., a single attendant) in the treatment setting at a time or for the duration of the psychoactive therapy session.

During the psychoactive therapy session, the treatment setting (e.g., the patient and/or the attendant) is monitored by a remote monitor, who is not present in the treatment setting. In some instances, the treatment setting comprises a video recording device. In some instances, the treatment setting comprises an audio recording device. In some instances, the remote monitor is on-site (e.g., in another room, e.g., not visible to the patient). In some instances, the remote monitor is off-site (e.g., in another building, e.g., on another facility or campus, such as a central monitoring station). For example, the remote monitor can monitor multiple treatment settings, regardless of proximity there between. In some instances, the remote monitor is responsible for monitoring multiple treatment settings simultaneously. In some embodiments, the remote monitor receives information (e.g., simultaneously or non-simultaneously) from 1-1,000 treatment settings, 2-750 treatment settings, 3-500 treatment settings, 4-200 treatment settings, 5-100 treatment settings, 6-50 treatment settings, 7-40 treatment settings, 8-30 treatment settings, 9-25 treatment settings, or 10-20 treatment settings (e.g., from 1-5 treatment settings, 2-8 treatment settings, 5-10 treatment settings, 10-20 treatment settings, 20-30 treatment settings, 30-40 treatment settings, 40-50 treatment settings, 50-100 treatment settings, 100-200 treatment settings, or more than 200 treatment settings, e.g., 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 160, 170, 180, 190, 200, or more treatment settings).

The remote monitor can be in communication (e.g., by a secure line of communication) with the one or more attendants for direct communication. Thus, the attendant can summon the monitor to the treatment setting to respond to any stimulus (e.g., including events not derived from session data). For example, a remote monitor may be summoned by the attendant to respond for technical support or temporary coverage, e.g., for bathroom breaks. In some embodiments, the line of communication between the attendant and monitor is a two-way line of communication (e.g., a secure two-way line of communication). In some instances, the remote monitor is not a physician (e.g., a psychiatrist). In other instances, the remote monitor is a physician (e.g., a psychiatrist). In instances in which the remote monitor is not a physician (e.g., a psychiatrist), the remote monitor may have access to a physician (e.g., a psychiatrist) to alert him or her of any pertinent information (e.g., session data (e.g., a patient response metric) necessitating assessment or intervention). In some instances, the remote monitor is in secure communication with the physician (e.g., via a secure line of communication, such as an encrypted messaging program). Additionally or alternatively, the remote monitor can summon clinical support from a non-physician (e.g., a non-physician having a clinical certification and/or a non-physician clinical professional specifically trained to address adverse patient response), e.g., to communicate directly with the attendant and/or patient (e.g., through intervention of the psychoactive therapy session).

### Session Data

Session data of the present methods and systems includes digitally recorded data and manually recorded data. Digitally recorded data includes video and/or audio data. Digitally recorded data can also be wearable sensor data (e.g., including acceleration data, movement data, or biometric data (e.g., heart rate, breathing characteristics, body temperature, EEG data, etc.)). In some embodiments, the session data is data obtained using biosensors, as described in International Publication No. WO 2019/079742, which is incorporated herein by reference in its entirety.

In some embodiments, the digitally recorded data comprises infrared sensor data. In some embodiments, the patient response metric comprises a measure of movement throughout the treatment setting derived from the digitally recorded data. In some embodiments, a high measure of movement throughout the treatment setting indicates an adverse patient response to the psychoactive therapy session. In some embodiments, the adverse patient response to the psychoactive therapy session is indicated by a high measure of movement throughout the treatment setting combined with a low measure of sudden motor movements.

In some embodiments, the digitally recorded data comprises EEG sensor data. Electrophysiological techniques can be used to investigate drug-induced changes in neural oscillations using, e.g., spectral power analyses, which can provide information regarding the contribution of neurons oscillating synchronously at particular frequencies to the measured signal (see Ward, L. M., Trends Cogn. Sci. 7, 553-559 (2003)). Oscillations within the human brain represent the rhythmic activity of neurons and span a frequency range from 0.05 to 500 Hz. The power spectrum of an EEG time series is most often calculated from a discrete fast Fourier transform which generates a complex value at each frequency whose modulus is reflective of amplitude, with the square of this coefficient giving the spectral power—a measure of the energy at each particular frequency. Neural oscillations are considered to be essential for the temporal coordination of activity between and within the various functional networks of the brain and are indicative of global state changes within the brain. Oscillatory activity measured at rest is believed to be reflective of the intrinsic functioning of networks and can provide an index of pharmacological or pathological modulation of functioning. While the generation of neural oscillations in each of the frequency bands require the synchronized activity of large populations of neurons, the generative mechanisms of oscillations in each frequency band are distinct and, as such, provide information regarding diverse neural processes. Using the methods described herein, changes in the power of different bands of EEG signals in a subject (e.g., gamma greater than 30 (Hz), beta (13-30 Hz), alpha (8-12 Hz), theta (4-8 Hz), and/or delta (less than 4 Hz)) can be used to monitor a subject's response to treatment, and when brain function has returned to normal post-treatment. In some embodiments, the subject undergoing the drug therapy experiences an observable decrease in alpha band spectral power and/or and increase in gamma band spectral power while the subject is under the influence of the drug. In some embodiments, the subject undergoing the drug therapy experiences an observable higher spontaneous signal diversity, even when controlling for spectral changes, while the subject is under the influence of the drug. Measures of entropy and Lempel-Ziv complexity both capture the diversity of a signal.

Manually recorded data includes answers by the patient to questions, e.g., questions asked by an attendant or other verbal or nonverbal communication by the patient to the attendant. Manually recorded data also includes observations by the attendant, including patient behavior. In some embodiments, the session data includes a rating of unresponsiveness, which may indicate an adverse response, such as distress and/or anxiety.

Either digitally recorded session data and manually recorded session data can include behavioral samples and/or language samples, providing behavior characteristics and/or language characteristics, respectively, such as those described in International Publication No. WO 2019/079742, which is incorporated herein by reference in its entirety.

For example, in some embodiments, the session data (e.g., digitally or manually recorded session data) includes a measure of conversation between the patient and the attendant (e.g., volume, frequency of exchange, length of pauses between words or phrases, etc.). In some embodiments, the session data (e.g., digitally or manually recorded session data) includes a measure of anxiety and/or paranoid ideation. In some embodiments, a high measure of anxiety and/or paranoid ideation indicates an adverse patient response to the psychoactive therapy session.

### Language Sample Acquisition

A language sample can be an audio sample (e.g., a verbal sample) and/or text sample (e.g., a typed or transcribed sample (e.g., a transcription from a verbal sample, e.g., by an attendant)). Language samples can be passively acquired (e.g., recorded, e.g., from conversations between the patient and attendant). In some embodiments, an attendant elicits the language sample from the patient, e.g., by administering a questionnaire, interview, or survey. Ecological Momentary Intervention or Ecological Momentary Assessment (EMA) may be administered (e.g., questions such as “how happy do you feel right now?” and “how stressed do you feel?”). Exemplary questionnaires include suicidality questionnaires, and psychopathology questionnaires. Mood questionnaires useful as part of the methods and systems of the invention include the Profile or Mood States (POMS), the Positive and Negative Affect Schedule (PANAS), and equivalent variants thereof. Suicidality questionnaires useful as part of the methods and systems of the invention include the Columbia Suicide Severity Rating Scale (C-SSRS) and equivalent variants thereof. Psychopathology questionnaires useful as part of the methods and systems of the invention include psychopathology questionnaires configured to assess hypomania or mania (e.g., the Hypomania/Mania Symptom Checklist (HCL-32), the Clinician-Administered Rating Scale for Mania (CARS-M), or the Young Mania Rating Scale (YMRS)) and psychopathology questionnaires configured to assess psychosis (e.g., the psychosis screening questionnaire, the Schizophrenia Test and Early Psychosis Indicator (STEPI), or the Cognitive Biases Questionnaire for psychosis (CBQp). Additionally or alternatively, any suitable EMA may be administered as part of the methods and systems of the invention provided herein.

One or more open ended questions or commands may be used to elicit a language sample, e.g., as an open-ended interview, to elicit free speech, which has been shown to provide a suitable source for predictive language characteristics. Exemplary methods thereof are described, for example, by Bedi et al (npj Schizophrenia 2015, 1:15030), incorporated herein by reference in its entirety.

In some embodiments, active acquisition of a language sample (e.g., an audio sample) involves eliciting speech, for example, as part of a dream report, a description of an image (e.g., as part of a thematic apperception test), or as a neutral text reading.

A language sample may be of any size or length suitable to provide one or more speech characteristics. For example, an audio language sample (e.g., an audio recording) can be from 1 millisecond to 120 minutes in length. In general, an acoustic analysis requires a shorter audio sample than a semantic analysis. In some embodiments, a sample useful for acoustic analysis is from 1 millisecond to 120 minutes in length (e.g., from 1 millisecond to 10 milliseconds, from 10 milliseconds to 20 milliseconds, from 20 milliseconds to 30 milliseconds, from 30 milliseconds to 40 milliseconds, from 40 milliseconds to 50 milliseconds, from 50 milliseconds to 100 milliseconds, from 100 milliseconds to 500 milliseconds, from 500 milliseconds to 1 second, from 1 second to 10 seconds, from 10 seconds to 30 seconds, from 30 seconds to 1 minute, from 1 minute to 10 minutes, from 10 minutes to 30 minutes, or from 30 minutes to 120 minutes in length).

In some embodiments, a sample useful for semantic analysis is from 5 seconds to 120 minutes in length (e.g., from 10 seconds to 90 minutes, from 20 seconds to 60 minutes, from 30 seconds to 45 minutes, from 1 minute to 30 minutes, or from 5 minutes to 15 minutes in length; e.g., from 10 seconds to 20 seconds, from 20 seconds to 30 seconds, from 30 seconds to 1 minute, from 1 minute to 5 minutes, from 5 minutes to 10 minutes, from 10 minutes to 20 minutes, from 20 minutes to 30 minutes, from 30 minutes to 60 minutes, from 60 minutes to 90 minutes, or from 90 minutes to 120 minutes in length). In some embodiments, an audio language sample (e.g., an audio recording) can be less than 60 minutes (e.g., less than 50 minutes, less than 45 minutes, less than 40 minutes, less than 35 minutes, less than 30 minutes, less than 25 minutes, less than 20 minutes, less than 15 minutes, less than 10 minutes, less than 5 minutes, less than 4 minutes, less than 3 minutes, less than 2 minutes, less than 60 seconds, less than 50 seconds, less than 40 seconds, less than 30 seconds, less than 20 seconds, or less than 10 seconds).

In some embodiments, a language sample (e.g., an audio sample (e.g., a verbal sample) and/or text sample (e.g., a typed or transcribed sample (e.g., a transcription from a verbal sample)) comprises from 20 words to 10,000 words (e.g., from 30 words to 8,000 words, from 50 words to 5,000 words, from 100 words to 2,500 words, or from 500 to 1,000 words; e.g., from 20 words to 30 words, from 30 words to 40 words, from 40 words to 50 words, from 50 words to 75 words, from 75 words to 100 words, from 100 words to 200 words, from 200 words to 500 words, from 500 words to 1,000 words, from 1,000 words to 2,500 words, from 2,500 words to 5,000 words, from 5,000 words to 7,500 words, or from 7,500 words to 10,000 words). In some embodiments, a language sample comprises less than 10,000 words (e.g., less than 9,000 words, less than 8,000 words, less than 7,000 words, less than 6,000 words, less than 5,000 words, less than 4,000 words, less than 3,000 words, less than 2,000 words, less than 1,000 words, less than 900 words, less than 800 words, less than 700 words, less than 600 words, less than 500 words, less than 400 words, less than 300 words, less than 200 words, less than 100 words, less than 90 words, less than 80 words, less than 70 words, less than 60 words, or less than 50 words).

### Behavioral Sample Acquisition

Various behavioral characteristics can be derived from one or more behavioral samples. In some instances, a behavioral sample is a measure of social behavior and/or physical activity (e.g., mobility, physiology, or other actions (e.g., psychomotor activity)). In some instances, a behavioral sample is passively acquired. In such instances, systems of the invention may feature acquisition programs that constantly or intermittently acquire data. Passive acquisition can occur at predetermined time points and/or for predetermined durations. In other instances, passive acquisition of behavioral samples can be initiated manually, e.g., by an attendant.

In other instances, a behavioral sample detected by a sensor. For example, physical activity of an individual may be detected or monitored by a sensor. For example, physical sensors include any device able to detect physical activity or characteristics (e.g., mobility, physiology, and/or motion, e.g., psychomotor activity), including video sensors (e.g., video cameras), motion sensors (e.g., passive infrared sensors, ultrasonic sensors, microwave sensors, or tomographic sensors), GPS, accelerometers (e.g., as part of a mobile device, such as a smartphone or smart wearable device), or biosensors (e.g., sensors that detect physiological characteristics, such as EEG signals, body mass, body temperature, heart rate, breathing characteristics (e.g., rate or depth), or blood characteristics (e.g., blood pressure, blood glucose levels, blood-drug concentration)).

Biosensors may be part of a mobile device, such as a smartphone, tablet, or wearable mobile device, such as a watch, bracelet, or necklace. Biosensors include sensors equipped with the capacity to detect the presence or level of one or more biomarkers (e.g., digital biomarkers), such as CO₂ levels (e.g., blood CO₂), glucose levels, expression of genes or proteins that correlate positively or negative with behavior.

A behavioral sample may be acquired for any suitable duration to provide one or more behavioral characteristics. In some embodiments, the behavioral sample is processed in real time, and the software continues to acquire the sample for the duration of time necessary to achieve a significant result. For example, the system may iteratively analyze the accumulating language sample until one or more characteristics or risk measures reaches a predetermined significance level, at which point the prompt may automatically conclude.

Biosensors can be configured to sense behavioral characteristics or to detect changes in biomarkers indicative of other characteristics. For example, biosensors can be configured to detect presence of a level of a target molecule present in a body sample obtained from the patient (e.g., a level of the psychedelic agent, a metabolite of the psychedelic agent, or another molecule that correlates positively or negatively with the level of the psychedelic agent in the patient). Accordingly, biosensors configured for use in the present methods can allow an access module (or remote monitor) to monitor the amount of a psychedelic agent in a patient at any one or more times, thereby informing decisions regarding dosing (e.g., whether to adjust a dose amount or frequency) and retreatment (e.g., whether a patient should be retreated or the frequency of retreatment). Biomarkers obtained by such biosensors can be referred to as “digital biomarkers.”

Additionally or alternatively, digital biomarkers can inform a patient response metric, such as an adverse response, such as a risk of having or developing a disorder (e.g., paranoid ideation, propensity towards paranoid thinking, paranoid personality disorder, personality disorders, intellectual disabilities (e.g., intellectual developmental disorder), bipolar disorder, depersonalization, dissociation, derealization, hallucinogen-psychoactive abuse, hallucinogen-use disorders, hallucinogen-induced disorders (e.g., hallucinogen-persisting perception disorder (HPPD), or high-risk behavior)). Accordingly, a patient response metric (e.g., an adverse response) can be derived from any one or more digital biomarkers to inform decisions resulting from the methods of monitoring provided herein.

### Access Modules

Methods can be carried out by an access module that receives session data (e.g., raw session data and/or processed session data) from the psychoactive therapy session. Systems of the invention include one or more access modules. A single access module can be configured to receive session data from multiple treatment settings (e.g., multiple psychoactive therapy sessions).

In a simple embodiment, an access module serves as an intermediary between the data acquisition and the monitoring or intervention. For example, an access module can relay digitally recorded session data (e.g., in processed or unprocessed (i.e., raw) form) to a remote monitor via real-time streaming (e.g., a live video and/or audio stream of the psychoactive therapy session to a remote monitor).

In more complex embodiments, the access module includes a processor that processes session data. For example, the access module can receive the session data (e.g., raw session data) and processes the session data (e.g., to derive a patient response metric available to a remote monitor, physician, or clinical support staff). In some embodiments, the access module receives the session data (e.g., raw session data), processes the session data (e.g., to derive a patient response metric), and relays the processed session data to a remote monitor, physician, or clinical support staff.

The processor processes session data to derive a patient response metric that describes a patient's response, which can be a therapy-induced altered state of consciousness, distress, anxiety, paranoia, dread, and/or other psychoactive drug effects (e.g., acute psychoactive drug effects). In some embodiments, a patient response metric discriminates between a psychopathology (e.g., bipolar disorder (e.g., bipolar mania) or schizophrenia) and a positive or adverse drug effect and serves as a predictor of treatment response. Patient response metrics include locomotion, unresponsiveness to a question, or other language or behavioral characteristics. Accordingly, processors of the methods and systems of the invention are configured for language and behavioral analytics known in the art or described herein.

In an exemplary embodiment, a processor receives video session data and derives a patient response metric by processing all or a portion of the video session data to obtain a measure of exploratory behavior according to known methods, such as those described in Perry et al., Arch. Gen. Psychiatry 2009, 66(10): 1072-1080 (e.g., by an amount of motor activity, sequential structure of the motor activity, and/or exploration of novel stimuli). Increases in such patient response metrics, relative to a reference level, indicate that a patient has, or may be developing, a psychopathology, such as bipolar mania or schizophrenia. Thus, in some embodiments, access modules include processors configured to quantify behavior using an open field paradigm (e.g., a human behavioral pattern monitor) and image processing software.

In some embodiments, the patient response metric comprises a measure of conversation (e.g., volume, frequency of exchange, length of pauses between words or phrases, etc.) between the patient and the attendant. In some embodiments, a lengthy inaudible conversation between the patient and the attendant indicates a deviation from protocol. In some embodiments, the length of inaudible conversation is at least 10 seconds, at least 15 seconds, at least 20 seconds, at least 30 seconds, at least 45 seconds, at least 60 seconds, at least two minutes, at least three minutes, at least four minutes, at least five minutes, at least six minutes, at least seven minutes, at least eight minutes, at least nine minutes, or at least 10 minutes long (e.g., between 10 seconds and 10 minutes, between 15 seconds and 10 minutes, between 30 seconds and 10 minutes, between 45 seconds and 10 minutes, between 60 seconds and 10 minutes, between two minutes and 10 minutes, between three minutes and 10 minutes, between four minutes and 10 minutes, or between five minutes and ten minutes long).

In some embodiments, the patient response metric comprises a measure of anxiety and/or paranoid ideation (e.g., derived from manually recorded data or digitally recorded data). In some embodiments, a high measure of anxiety and/or paranoid ideation indicates an adverse patient response to the psychoactive therapy session. In some embodiments, the method further includes alerting clinical support of the adverse patient response.

In some embodiments, the access module receives and/or processes behavior characteristics and/or language characteristics, as described in International Publication No. WO 2019/079742, which is incorporated herein by reference in its entirety.

### Language Sample Processing

The methods and systems provided herein feature automated language analysis to process and analyze one or more language samples obtained from a patient. In some embodiments, the invention provides methods and systems that utilize automated speech analysis to determine a patient response metric (e.g., an adverse response). An exemplary automated speech analysis procedure that can predict onset of psychosis is described by Bedi (Bedi et al., npj Schizophrenia 2015, 1:15030), which is incorporated herein by reference in its entirety.

In another embodiment, the invention provides methods and systems that utilize automated acoustic analysis to determine a patient response metric. An exemplary automated acoustic analysis procedure that can predict onset of psychosis is described by Faurholt-Jepsen (Faurholt-Jepsen et al., Trans/Psychiatry 2016, 6: e856), which is incorporated herein by reference in its entirety. Additionally or alternatively, methods described in Agurto et al., Neuropsychopharmacol. 2020: 1-10 (incorporated herein by reference in its entirety) can be readily adapted for determining a patient response metric for use in the methods and systems described herein.

In general, language analysis begins with an input of a language sample (e.g., an audio sample (e.g., a verbal sample) and/or text sample (e.g., a typed or transcribed sample (e.g., a transcription from a verbal sample)). Pre-processing steps may be employed, such as transcribing verbal speech from an audio file into text. Additional pre-processing steps can be performed as described by Bedi, for example, using available tools, such as the Natural Language Toolkit (http://www.nltk.org). For example, punctuation may be removed, and phrases may be parsed. In some instances, words can be converted to the roots from which they are inflected, or lemmatized, e.g., using NLTK Wordnet lemmatizer. In some embodiments of the methods provided herein, pre-processed data includes a list of words that have been lemmatized, parsed into phrases, converted to lower case, maintained in their original order, and/or punctuation removed. Voice features can be extracted as described in Faurholt-Jepsen, for example, using available tools, such as openSMILE toolkit (Eyben F, et al., Proceedings of ACM Multimedia: Firenxe, Italy, 2010).

After any necessary pre-processing steps are performed, one or more characteristics of the language sample can be derived. Characteristics that can be derived from a language sample (e.g., a typed or transcribed sample (e.g., a transcription from a verbal sample)) include semantic coherence, syntactic complexity, comprehension, lexicon depth, lexicon breadth, or semantic proximity to one or more concepts related to an influence of a psychedelic agent (e.g., as described in the 5D-ASC rating scale). In general, a low measure of semantic coherence (e.g., similarity among pairs of consecutive phrases, or pairs of phrases separated by an intervening phrase) is positively correlated with risk of developing psychosis. Similarly, a low measure of syntactic complexity may be positively correlated with risk of developing psychosis; a low measure of lexicon depth may be positively correlated with risk of developing psychosis; and/or a low measure of lexicon breadth may be positively correlated with risk of developing psychosis. Less verbosity (e.g., a maximum number of words per phrase) can also be indicative of development of psychosis.

Language characteristics that can correlate with hypomania or mania include logorrhea (e.g., excess wordiness, higher Type/Token ratio, or incoherence), and recursiveness (e.g., returns to the same topic). Thus, in some instances, a measure of logorrhea and/or recursiveness can be used to derive a measure of risk of exacerbating or precipitating hypomania or mania.

As a derivation of any of the characteristics described herein is unique to its experimental circumstances (i.e., it is qualitative and relative), any of the preceding methods of deriving a characteristic from a language sample may result in a “measure” of that characteristic, which herein encompasses any value into which the quantity of the characteristic factors. For example, in some embodiments, semantic coherence may be weighted to a greater extent than syntactic complexity in an algorithm for calculating risk of psychosis.

Audio samples may provide further language characteristics from which a measure of risk of psychosis, hypomania, or mania can be derived. For example, speech quantity, rate, and fluctuation in pitch can be correlated with a high risk of developing hypomania or mania.

Acoustic features, such as pitch frequency F0, zero-crossing rate (ZCR), harmonics to noise ratio (HNR), mel-frequency cepstral coefficients (MFCC), and root mean squared (RMS) frame energy can be associated with prodromal psychosis, hypomania, or mania. Other acoustic features and their predictive value of psychosis, hypomania, and/or mania known in the art are contemplated for use as part of the methods and systems described herein. Such acoustic features can be characterized or summarized using known methods, including spectral mapping and statistical functions, such as means, standard deviations, ranges, kurtosis energies, extremes, moments, segments, peaks, linear and quadratic regressions and coefficients thereof, percentiles, durations, onsets, zero-crossings, modulation spectra, and composites thereof.

In some embodiments, the methods and systems of the invention feature a toolkit (e.g., openSMILES), which can be run directly, for example, on a mobile device (e.g., a smartphone or tablet) in real time, as the audio sample is acquired. In some instances, raw or processed data can be input into a model that is user-specific (e.g., compared with baseline values of the same user) or user-independent (e.g., compared with a predetermined threshold or a composite of values obtained by other users).

Additional methods of measuring, processing, and characterizing any of the aforementioned acoustic features are known in the art and describe, for example, in Vanello et al., Conf Proc IEEE Eng Med Biol Soc 2012. 2012:2104-2107 and Karam et al., Proceedings of the IEEE International Conference on Acoustics, Speech, and Signal Processing (Conference) 2014:4858-4862, both of which are incorporated by reference in their entireties.

Methods of deriving characteristics from language samples are known in the art. For example, latent semantic analysis (LSA) can be used to analyze language samples to derive characteristics involved in psychosis, as described in Landauer et al. (Psychol Rev 1997, 104:211-240) and Bedi, both of which are incorporated by reference. Alternative methods for deriving characteristics correlating with risk of developing psychosis, hypomania, or mania are described, for example, in Bedi et al. (Neuropsychopharmacology 2014, 39, 2340-2348) and Mota et al. (Scientific Reports 2014, 3691).

In some embodiments of any of the methods described above, a measure of any characteristic of a language sample can be derived using a machine learning algorithm, as described, for example, in Bedi. In some embodiments, a supervised or unsupervised cluster model can be used, e.g., to classify characteristics among a population of candidates or patients, or to compare one or more characteristics of a candidate or patient with a those of a reference population. The measure of risk may be determined using a Random Forest classifier or a within-patient Naïve Bayes classifier.

A patient response metric be determined using one or more characteristics of a language sample obtained using any of the methods described herein. As part of this step, in some embodiments of the methods and systems disclosed herein, a machine learning algorithm can be utilized. Similarly, a supervised or unsupervised cluster model can be used, e.g., to classify a measure of risk of a candidate or patient, e.g., as high risk or low risk.

In any of the preceding methods of language analysis, a measure of a characteristic can be determined based on comparison with a reference characteristic (i.e., a characteristic derived from one or more individuals of a reference population). In any of the methods disclosed herein, a change in a language characteristic may be identified by comparison with one or more prior language samples, such as a comparison to a baseline measure (e.g., at a time point in which the candidate or patient did not have prodromal or symptomatic psychosis, mania, or hypomania).

In some embodiments, a patient response metric is determined by a composite score of two or more characteristics of a language sample (e.g., a text sample and/or an audio sample). For example, a patient response metric can be determined by considering one, two, three, four, five, or six of any of the characteristics selected from the group consisting of semantic coherence, syntactic complexity, comprehension, lexicon depth, or lexicon breadth.

In some embodiments, session data comprising a language sample is processed in real time, and access module continues to record session data for the duration of time necessary to achieve a significant result. For example, the access module may iteratively analyze the accumulating language sample until one or more patient response metrics reaches a predetermined significance level, at which point the process may automatically conclude.

### Behavioral Sample Processing

The methods and systems provided herein feature behavioral analytics to process and analyze one or more behavioral samples obtained from a patient. In some embodiments, the patient response metric comprises a measure of movement throughout the treatment setting derived from the digitally recorded data. In some embodiments, a high measure of movement throughout the treatment setting indicates an adverse patient response to the psychoactive therapy session. In some embodiments, a high measure of movement throughout the treatment setting, in combination with a low measure of sudden motor movements, indicates an adverse patient response to the psychoactive therapy session.

In an exemplary embodiment, a processor receives video session data and derives a patient response metric by processing all or a portion of the video session data to obtain a measure of exploratory behavior according to known methods, such as those described in Perry et al., Arch. Gen. Psychiatry 2009, 66(10): 1072-1080 (e.g., by an amount of motor activity, sequential structure of the motor activity, and/or exploration of novel stimuli). Increases in such patient response metrics, relative to a reference level, indicate that a patient has, or may be developing, a psychopathology, such as bipolar mania or schizophrenia. Thus, in some embodiments, access modules include processors configured to quantify behavior using an open field paradigm (e.g., a human behavioral pattern monitor) and image processing software.

A patient response metric can be determined using one or more characteristics of a behavioral sample obtained from session data using any of the methods described herein. As part of this step, in some embodiments of the methods and systems disclosed herein, a machine learning algorithm can be utilized. Supervised or unsupervised cluster models can be used.

### System Architecture

The systems and methods of this invention can include or be implemented using any suitable processing system(s). Suitable processing systems include a computer based server (e.g., a remote server), a grid-computer system, a mainframe computer system, or a cloud-based computing platform. A processing system can be powered by any suitable computer processor (e.g. Intel or AMD processors), running operating systems such as Windows, Linux, Unix, or other standard operating systems. A server may have several gigabytes of random access memory. Servers can be connected to mass storage devices capable of storing gigabytes of data. The system and methods of the invention can be implemented in the form of software, such as database management software (e.g. SQL software which may run under popular database management programs such as MySQL or other systems such as Oracle), web server software such as Apache, programming languages for dynamic web pages such as PHP, Perl, Python, as well as other web application framework languages and systems languages such as Ruby on Rails, and the like. A server can be connected to suitable network, such as the internet, often by a high bandwidth connection.

In some embodiments, a network-connected computerized database contains a record of clinical support features, such as contact information for specialized treatment facilities, pharmacies, physicians, emergency personnel, and/or other support services. A system of the invention may include a software application (e.g., a mobile-device application) that accesses such a database conditionally or automatically to send one or more notifications, alerts, reports, or other information to a third party (e.g., to a computing platform (e.g., a remote database) or a clinical professional) for storage or analysis.

Various patient information (e.g., patient response metrics) can be stored on a processing system and/or shared with a third party, including data obtained from a screening session, such as raw data from a language sample (e.g., audio data, or text data), partially processed data obtained from a language sample (e.g., representation, summarization, or integration of a semantic analysis), a characteristic derived from processing a language sample (e.g., a semantic coherence measure, a syntactic complexity measure, a comprehension measure, a lexicon depth measure, or a lexicon breadth measure), a measure derived from one or more or such characteristics (e.g., a composite score derived from two or more of such characteristics (e.g., a composite of semantic coherence and syntactic complexity, or a measure of risk), or a relationship between any of the aforementioned data, e.g., obtained at different screening sessions, e.g., at different time points.

Behavioral data can also be stored on a processing system and/or shared with a third party. In some instances, such data includes social behavior data, mobility data, motion data, or any combination thereof.

Additionally or alternatively, personal identification information, medical records, and any messages or notes provided by the patient can be sent with a notification, alert, or report to a third party.

In some embodiments, the server database contains sufficient audio-video link information to establish audio and video communication between the candidate or patient and a clinician. For example, the server and database may contain both the internet address information for the practitioner, patient, and any third parties as needed, and also act to relay the data packets between the parties. Alternatively, the server and database may contain address links, such as, for example, for Skype or other online video conferencing systems enabling the patient, healthcare practitioner, and third parties to communicate by third party messaging systems. In general, in order to ensure quality and a consistent user interface, often the server will both present the telemedicine user interface (e.g. present one or more web pages for telemedicine applications) in addition to relaying the audio and video data packets. Accordingly, a telemedicine session can be suitably encrypted.

In any of the embodiments described above, the system can be configured to adhere to health-related privacy laws (e.g., HIPAA, GDPR). For example, systems can be configured to privatize and/or anonymize individual data according to encryption protocols.

### Methods of Intervening

In another aspect, provided herein is a method of intervening in a psychoactive therapy session. The method includes: (i) receiving session data via an access module (e.g., uploading session data onto an access module), wherein the session data is transmitted to the access module from the psychoactive therapy session, wherein the psychoactive therapy session is conducted in a treatment setting comprising a patient and an attendant, and wherein no physician (e.g., psychiatrist) is present in the treatment setting; (ii) transferring the session data from the access module to a remote monitor; (iii) identifying an aberrance in the session data; and (iv) summoning clinical support to the treatment setting, thereby intervening in the psychoactive therapy session. In some embodiments, the aberrance is misconduct by the attendant. In some embodiments, the method further includes processing the session data (e.g., within the access module, e.g., within a processor) to derive a patient response metric. In some embodiments, the method further includes transferring the patient response metric from the access module to the remote monitor. In some embodiments, the aberrance is an adverse patient response to the psychoactive therapy session. In some embodiments, the aberrance is a deviation from protocol (e.g., clinical misconduct).

In some embodiments, intervention includes steps of escalation. For example, upon notification of an aberrance to a remote monitor (e.g., by direct observation or by the attendant alerting the remote monitor (e.g., via a communication line (e.g., a secure and/or two-way communication line)), the remote monitor chooses to alert another staff member or a psychiatrist, either of whom could then intervene (either remotely or locally). In certain instances, where the remote monitor alerts another clinical staff member (e.g., a non-physician), the other clinical staff member then chooses whether to summon a physician (e.g., a psychiatrist) for intervention (either remotely or locally).

In another aspect, the invention is method of intervening in a psychoactive therapy session, the method comprising: (i) receiving session data via an access module (e.g., uploading session data onto an access module), wherein the session data is transmitted to the access module from the psychoactive therapy session, wherein the psychoactive therapy session is conducted in a treatment setting comprising a patient and an attendant, and wherein no physician (e.g., no psychiatrist) is present in the treatment setting; (ii) within the access module (e.g., within a processor), processing the session data to derive a patient response metric indicative of a patient response to the psychoactive therapy session; (iii) identifying an aberrance based on the patient response metric; and (iv) summoning clinical support to the treatment setting, thereby intervening in the psychoactive therapy session. In some embodiments, the clinical support includes a physician (e.g., a psychiatrist). Additionally or alternatively, the clinical support can include one or more non-physician clinical professionals (e.g., one or more non-physician clinical professionals specifically trained to address adverse patient response). For example, non-physician clinical professionals can be trained and/or certified in approaches for addressing adverse responses, such as administration of benzodiazepine, supportive talk, etc.

In some embodiments, the method further includes transferring the patient response metric from the access module to a remote monitor. In some embodiments, the method further includes transferring unprocessed session data from the access module to the remote monitor. In some embodiments, the session data comprises digitally recorded data, such as video and/or audio data. Additionally or alternatively, the digitally recorded data comprises wearable sensor data (e.g., including acceleration data, movement data, or biometric data (e.g., heart rate, breathing characteristics, body temperature, EEG data, etc.).

In some embodiments of any of the preceding aspects, the session data comprises manually recorded data provided by the attendant (e.g., a rating of unresponsiveness). Additionally or alternatively, the manually recorded data comprises a patient self-report.

In some embodiments, the patient response metric comprises a measure of anxiety and/or paranoid ideation (e.g., derived from manually recorded data or digitally recorded data). In some embodiments, a high measure of anxiety and/or paranoid ideation indicates an adverse patient response to the psychoactive therapy session. In some embodiments, the method further includes alerting clinical support of the adverse patient response.

In another aspect, provided is a method of conducting a psychoactive therapy session, the method comprising: (i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; (ii) within the access module, processing the session data to derive a first patient response metric indicative of a psychoactive drug effect, and processing the session data to derive a subsequent patient response metric indicative of the psychoactive drug effect; (iii) comparing the first patient response metric with the subsequent patient response metric; and (iv) based on the comparison of (iii), determining whether to dismiss the patient from the treatment setting. In some embodiments, the method further includes dismissing the patient from the treatment setting. In some embodiments, the access module comprises a processor, and wherein the processor performs the comparison.

### Methods of Generating and Editing a Database

In another aspect, the invention includes a method of editing session data from a psychoactive therapy session. Such methods can be used for training purposes, by isolating a relatively small pertinent dataset from the larger dataset (including extraneous (less pertinent) data) to illustrate particular patient-attendant interactions, patient behavior, or other patient response. Such methods include (i) receiving session data via an access module, wherein the session data is transmitted to the access module from the psychoactive therapy session; and (ii) within the access module: (a) processing the session data comprising a pertinent dataset and an extraneous dataset to derive a patient response metric indicative of a patient response to the psychoactive therapy session, wherein the patient response metric is preferentially based on the pertinent dataset; and (b) producing an edited dataset by isolating the pertinent dataset from the extraneous dataset. Methods may further include using the edited dataset for debriefing an attendant, physician, or other clinical support staff, or for future training of an attendant, physician, or other clinical support staff.

In another aspect, the invention includes a method of generating a patient response database. Such methods can be used to normalize conditions across treatment settings and/or to normalize the processing of session data, e.g., to a global database. Such methods include: (i) via an access module, receiving session data from a first psychoactive therapy session and session data from a second psychoactive therapy session, wherein the first and second psychoactive therapy sessions are each conducted in a treatment setting comprising a patient and an attendant, wherein no physician is present in the treatment setting during either of the first and second psychoactive therapy sessions; (ii) within the access module: (a) processing the session data from the first psychoactive therapy session to derive a first patient response metric indicative of a first patient response to the psychoactive therapy session; and (b) processing the session data from the second psychoactive therapy session to derive a second patient response metric indicative of a second patient response to the psychoactive therapy session; and (iii) recording the first patient response metric and the second patient response metric to a computer-readable medium, thereby generating a patient response database. In some embodiments, the method further includes deriving an average patient response from the first patient response and the second patient response. In some embodiments, the invention further includes receiving session data from a third, fourth, fifth, sixth, and/or seventh psychoactive therapy session each conducted in a treatment setting comprising a patient and an attendant, wherein no physician is present in the treatment setting during any of the psychoactive therapy sessions; within the access module: processing the session data from the third, fourth, fifth, sixth, and/or seventh psychoactive therapy sessions to derive a third, fourth, fifth, sixth, and/or seventh patient response metric indicative of a third, fourth, fifth, sixth, and/or seventh patient response to the psychoactive therapy session; and (b) integrating the session data from the third, fourth, fifth, sixth, and/or seventh psychoactive therapy sessions with the session data from the first and second psychoactive therapy sessions to derive a third, fourth, fifth, sixth, and/or seventh patient response metric indicative of a third, fourth, fifth, sixth, and/or seventh patient response to the psychoactive therapy session; and (iii) recording the third, fourth, fifth, sixth, and/or seventh patient response metric to a computer-readable medium. It will be readily understood that such methods are scalable and can integrate session data from any number of therapy sessions and patients.

### Therapies

The invention includes methods of administering a psychoactive therapy session through any of the methods and systems described herein. Using the methods and systems of the invention, a psychoactive agent (e.g., a psychedelic agent) can be administered on an in-patient basis or an out-patient basis. In some instances, a psychedelic agent is administered to a patient by a clinical professional. In some embodiments, a perceptible dose of a psychedelic therapy is administered (e.g., a dose of greater than about 0.1 μg/kg, greater than about 0.5 μg/kg, greater than about 1.0 μg/kg, greater than about 5.0 μg/kg, greater than about 10 μg/kg, greater than about 20 μg/kg, greater than about 50 μg/kg, greater than about 100 μg/kg, greater than about 200 μg/kg, greater than about 500 μg/kg, greater than about 1.0 mg/kg, greater than about 5.0 mg/kg, greater than about 10 mg/kg, greater than about 50 mg/kg, or greater than about 100 mg/kg body weight, e.g., from about 0.1 μg/kg to about 0.5 μg/kg, from about 0.5 μg/kg to about 10 μg/kg, from about 1.0 μg/kg to about 5.0 μg/kg, from about 5.0 μg/kg to about 10 μg/kg, from about 10 μg/kg to about 50 μg/kg, from about 50 μg/kg to about 100 μg/kg, from about 100 μg/kg to about 500 μg/kg, from about 500 μg/kg to about 1.0 mg/kg, from about 1.0 mg/kg to about 10 mg/kg, from about 10 mg/kg to about 50 mg/kg, from about 50 mg/kg to about 100 mg/kg, or from about 100 mg/kg to about 500 mg/kg).

In some embodiments, a perceptible dose of psilocybin can be from 10 mg to 50 mg (e.g., from 10-25 mg, or from 25-50 mg, e.g., about 10 mg, about 15 mg, about 20 mg, about 25 mg, about 30 mg, about 35 mg, about 40 mg, about 45 mg, or about 50 mg). In some embodiments, a perceptible dose of LSD can be from 100 μg to 500 μg (e.g., from 100 μg to 200 μg, from 200 μg to 300 μg, from 300 μg to 400 μg, or from 400 μg to 500 μg, e.g., about 100 μg, about 150 μg, about 200 μg, about 250 μg, about 300 μg, about 350 μg, about 400 μg, about 450 μg, or about 500 μg).

In some instances, a perceptible dose of a psychedelic therapy is administered as an out-patient procedure, and the patient is monitored before release to ensure that any perceptible psychedelic effects (e.g., influences) have subsided. In this instance, the influence of the psychedelic therapy can be characterized at one or more (e.g., two, three, four, five, or more) time points following administration, e.g., to monitor its kinetics. For example, based on one or more patient metrics derived from session data, a measure of influence can be derived. In some embodiments, the patient is monitored shortly after administration of the psychedelic agent (e.g., from 1-10 minutes, from 10-20 minutes, from 20-30 minutes, or within 1 hour, e.g., at 1 minute, 2 minutes, 3 minutes, 4 minutes, 5 minutes, 10 minutes, 15 minutes, 20 minutes, 25 minutes, 30 minutes, 40 minutes, or 50 minutes) to determine an influence measure at or near the psychedelic agent's peak effect. This peak influence measure may be compared to a reference measure (e.g., a baseline measure obtained from the same patient, or a measure derived from a plurality of subjects characterized as having a low influence measure or a low risk of developing psychosis, hypomania, or mania, e.g., as determined using any of the methods described herein). A subsequent language sample may be taken after any period of time from administration in which a psychedelic influence may have subsided (e.g., from 1-72 hours, e.g., from 24-72 hours or from 36-48 hours after administration, e.g., from 1-2 hours, from 2-3 hours, from 3-4 hours, from 4-5 hours, from 5-6 hours, from 6-7 hours, from 7-8 hours, from 8-10 hours, from 10-12 hours, from 12-14 hours, from 14-16 hours, form 16-18 hours, form 18-20 hours, from 20-22 hours, from 22-24 hours, from 24-36 hours, from 36-42 hours, from 42-48 hours, from 48-60 hours, or from 60-72 hours after administration, e.g., about 24 hours, about 36 hours, about 48 hours, about 60 hours, or about 72 hours after administration). The degree to which a psychedelic influence subsides can be characterized, for example, by a decrease from a peak influence measure to a subsequent influence measure.

In some instances, one or more language samples are elicited following administration of a psychedelic agent as part of an out-patient procedure to provide a final evaluation of the psychedelic effects on the patient. In some embodiments, the language sample is elicited by a structured interview with an attendant.

Alternatively, a psychedelic influence may be determined to have subsided by an alternative characterization method, for example, by comparing to a reference measure (e.g., a baseline measure obtained from the same patient, or measure derived from a plurality of subjects characterized as having a low influence measure or a low risk of developing psychosis, hypomania, or mania, e.g., as determined using any of the methods described herein). For example, an influence measure falling within a predetermined range (e.g., a range of error, e.g., a predetermined standard error) of a reference measure can used as a threshold below which a patient can be identified as having negligible or no residual psychedelic influence.

In some embodiments of the invention, a sub-perceptible dose of a psychedelic therapy is administered (e.g., a dose of less than about 100 μg/kg, less than about 75 μg/kg, less than about 50 μg/kg, less than about 25 μg/kg, less than about 10 μg/kg, less than about 7.5 μg/kg, less than about 5.0 μg/kg, less than about 2.0 μg/kg, less than about 1.5 μg/kg, less than about 1.0 μg/kg, less than about 0.5 μg/kg, less than about 0.1 μg/kg, or less, e.g., from about 0.1 μg/kg to about 0.5 μg/kg, from about 0.5 μg/kg to about 10 μg/kg, from about 1.0 μg/kg to about 5.0 μg/kg, from about 5.0 μg/kg to about 10 μg/kg, from about 10 μg/kg to about 50 μg/kg, or from about 50 μg/kg to about 100 μg/kg). Sub-perceptible doses may, in some circumstances, be administered as an out-patient procedure.

In any of the above contexts, a course of administration may be altered according to a result of any session data or derivation thereof. In some embodiments, a psychoactive therapy is suspended if a patient is identified as at risk of developing psychosis, hypomania, or mania. The invention also allows for a dosage modification, as appropriate, according to the patient's response to therapy.

### Patient Populations

The methods and systems of the present invention involve patients who are undergoing therapy for a condition (e.g., alleviation of symptoms of a condition) or improvement of mental or physical well-being (e.g., as a component of a wellness facility or retreat). In some embodiments, a patient has a psychological condition treatable by a psychedelic therapy. Psychological conditions treatable by a psychedelic therapy (e.g., a 5-HT2A receptor agonist (e.g., LSD, psilocybin, DOI, or (R)-DOI), an empathogenic agent, (e.g., MDMA), or a dissociative agent (e.g., ketamine)) include depression (e.g., major depression, melancholic depression, atypical depression, or dysthymia), an anxiety disorder (e.g., end of life anxiety, generalized anxiety disorder, panic disorder, social anxiety, post-traumatic stress disorder, acute stress disorder, obsessive compulsive disorder, or a social phobia), an addiction (e.g., a substance abuse or an eating disorder), a compulsive disorder (e.g., a primary impulse-control disorder or an obsessive-compulsive disorder), or a repetitive body-focused behavior (e.g., tic disorder or symptom thereof, such as Tourette's Syndrome, trichotillomania, nail-biting, temporomandibular disorder, thumb-sucking, repetitive oral-digital, lip-biting, fingernail biting, eye-rubbing, skin-picking, or a chronic motor tic disorder).

The methods and systems of the invention can be used with patients having a substance abuse problem, drug addiction, or other addictive behaviors. Addictive behaviors which can be treated using psychedelic therapy include food addiction, binge eating disorder, pathological gambling, pathological use of electronic devices, pathological use of electronic video games, pathological use of electronic communication devices, pathological use of cellular telephones, addiction to pornography, sex addiction, obsessive-compulsive disorder, compulsive spending, intermittent explosive disorder, kleptomania, pyromania, trichotillomania, compulsive over-exercising, and compulsive overworking. Drug addictions which can be treated using the methods of the invention include addictions to recreational drugs, as well as addictive medications. Examples of addictive agents include, but are not limited to, alcohol, e.g., ethyl alcohol, gamma hydroxybutyrate (GHB), caffeine, nicotine, cannabis (marijuana) and cannabis derivatives, opiates and other morphine-like opioid agonists such as heroin, phencyclidine and phencyclidine-like compounds, sedative hypnotics such as benzodiazepines, methaqualone, mecloqualone, etaqualone and barbiturates and psychostimulants such as cocaine, amphetamines and amphetamine-related drugs such as dextroamphetamine and methylamphetamine. Examples of addictive medications include, e.g., benzodiazepines, barbiturates, and pain medications including alfentanil, allylprodine, alphaprodine, anileridine benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofenitanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, OXYCONTIN®, oxymorphone, papaveretum, pentazocine, phenadoxone, phenomorphan, phenazocine, phenoperidine, piminodine, piritramide, propheptazine, promedol, properidine, propiram, propoxyphene sufentanil, tramadol, and tilidine.

Additionally or alternatively, patients described herein may have an anxiety disorder. Anxiety is broadly defined as a state of unwarranted or inappropriate worry often accompanied by restlessness, tension, distraction, irritability and sleep disturbances. This disproportionate response to environmental stimuli can hyperactivate the hypothalamic-pituitary-adrenal axis and the autonomic nervous system, resulting in somatic manifestation of anxiety, including shortness of breath, sweating, nausea, rapid heartbeat and elevated blood pressure. Anxiety disorders represent a range of conditions and as a result have been classified into multiple distinct conditions, including generalized anxiety disorder (GAD), panic disorder, social anxiety, post-traumatic stress disorder (PTSD), acute stress disorder (ASD), obsessive compulsive disorder (OCD), and social phobias. GAD is the most commonly occurring of the anxiety disorders and is characterized by excessive and persistent worries. In the general population the lifetime prevalence rate of GAD ranges from 4.1 to 6.6% with somewhat higher rates in women than in men. The individual with GAD worries about life events such as marital relationships, job performance, health, money, and social status. Individuals with GAD startle easily and may suffer from depression. Some of the specific symptoms of GAD include restlessness, motor tension, difficulty concentrating, and irritability. The severity of the symptoms over time may be linked to the changing nature of the environmental stressor. With increasing age, GAD symptoms become less severe. Panic disorder is a well-studied psychiatric condition that consists of multiple disabling panic attacks characterized by an intense autonomic arousal. In addition, heightened fear and anxiety states occur both during and between panic attacks. Approximately 3% of women and 1.5% of men have panic attacks. During a panic attack, the individual experiences multiple symptoms including light-headedness, a pounding heart and difficulty in breathing.

PTSD is a disorder characterized by intense fear and anxiety states that require psychiatric treatment. PTSD often results from exposure to a life threatening or traumatic event. Individuals with PTSD can have recurring thoughts of the terrifying event. Reenactment of the event varies in duration from a few seconds or hours to several days.

Additionally or alternatively, patients described herein may have an adjustment disorder, i.e., depression and/or anxiety associated with cancer or a terminal diagnosis (e.g., bereavement, grief (e.g., family/loved ones grief, complex grief (e.g., associated with adverse childhood experience)). In such embodiments, the methods and systems described herein can be used as part of a hospice care program. In such instances, the patient may be the hospice patient and/or the hospice patient's loved one (e.g., family member, spouse, etc.).

Additionally or alternatively, patients described herein may be undergoing neurorehabilitation. For example, patients may be undergoing neurorehabilitation for recovery from neurological injury (e.g., stoke, traumatic brain injury, spinal cord injury). In such embodiments, the methods and systems described herein can be used as part of a neurorehabilitation program, e.g., in which a 5-HT_{2A} receptor agonist, empathogen, or other psychoactive drug is administered.

In some instances of any of the methods and systems described herein, the patient is obese and is being treated for weigh loss (or behavioral change underlying the obese condition).

A psychedelic therapy can treat one or more symptoms (e.g., prodromal or somatic symptoms) of a psychological disorder. For example, a psychedelic therapy can treat a prodromal symptom of a depressive disorder (e.g., depressed mood, decreased appetite, weight loss, increased appetite, weight gain, initial insomnia, middle insomnia, early waking, hypersomnia, decreased energy, decreased interest or pleasure, self-blame, decreased concentration, indecision, suicidality, psychomotor agitation, psychomotor retardation, crying more frequently, inability to cry, hopelessness, worrying/brooding, decreased self-esteem, irritability, dependency, self-pity, somatic complaints, decreased effectiveness, helplessness, or decreased initiation of voluntary responses). Additionally or alternatively, a psychedelic therapy can treat a somatic symptom, e.g., a somatic symptom associated with a psychological disorder (e.g., chronic pain, anxiety disproportionate to severity of physical complaints, pain disorder, body dysmorphia, conversion, hysteria, neurological conditions without identifiable cause, psychosomatic illness, or pain management in relation to an existing physical condition).

### Complex Therapy

In some embodiments, the methods provided herein are performed in the context of an authorized treatment facility (e.g., a specialized treatment facility) configured to provide complex therapies to subjects in need thereof. Complex therapies may involve both pharmaceutical (e.g., psychedelic agent-based) and non-pharmaceutical treatments (e.g., behavioral therapy (e.g. cognitive behavioural therapy (CBT), brief behavioral activation for depression (BATD), talk therapy, existential therapy, and/or self-actualization therapy) designed according to a subject's specific needs. For example, methods provided herein enable a practitioner to determine whether a subject is likely to benefit from a psychedelic treatment and act accordingly. In many instances, a psychedelic treatment regimen may not be prescribed (and may be detrimental) outside the context of a specialized treatment facility in which a subject has access to adjunctive psychotherapy (e.g., behavioral therapy, existential, humanistic, or self-actualization therapy). Specialized treatment facilities can be configured to enhance the safety and efficacy of therapy (e.g., psychedelic therapy and/or complex therapy) through control and use of audio, visual, and other environmental factors. In general, specialized treatment facilities feature a staff that has training and expertise in administering and overseeing complex therapy, including psychedelic therapy and psychotherapy.

Treatment facilities in which psychedelic and/or complex therapies can be administered include other settings that are authorized to administer therapies including psychedelic therapies, adjunctive psychotherapies, and/or complex therapies. For example, authorized treatment facilities may be associated with a hospital, a mental health clinic, or a retreat center. Treatment facilities may be in-patient or out-patient facilities and may provide screening, evaluation, and follow-up services. In some embodiments, treatment facilities may be associated with a research facility/program.

A patient's response to therapy (e.g., psychedelic therapy, psychotherapy, and complex therapy) can be monitored and quantified using any suitable method known in the art according to the particular condition being treated.

### Psychoactive Agents

The invention includes methods and systems involving a patient who is undergoing treatment with a psychoactive agent (e.g., a psychedelic agent) or who is a candidate for treatment with a psychoactive agent (e.g., a psychedelic agent). Psychedelic agents useful as part of the invention include any compound capable of inducing an altered state of consciousness, i.e., a marked deviation in the subjective experience or psychological functioning of a normal individual from his or her usual waking consciousness. Psychedelic agents include 5-HT_{2A} agonists (e.g., lysergic acid diethylamide (LSD), empathogenic agents (i.e., serotonin (5-HT) releasing agents; e.g., 3,4-methylenedioxymethamphetamine (MDMA)), and dissociative agents (i.e., N-Methyl-D-aspartate (NMDA) receptor agonists; e.g., ketamine).

5-HT_{2A} agonists include psilocybin, LSD, DOI (±)-1-(2,5-dimethoxyphenyl)-2-aminopropane hydrochloride; (R)-DOI ((R)-1-(2,5-dimethoxy-4-iodophenyl)-2-aminopropane) (greater than 95% R enantiomer); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl)methylamine; ayahuasca; 3,4,5-trimethoxyphenethylamine (mescaline); 5-methoxy-N,N-dimethyltryptamine (5-meo-DMT); ibogaine; voacangine; noribogaine; coronaridine; 18-methoxycoronaridine (18-mc); methoxyethyl 18-MC; 18-methylaminocoronaridine (18-MAC); and 2-methoxyethyl-18-methoxycoronaridinate.

In some embodiments, the 5-HT_{2A} agonist of the invention includes a compound having the following chemical formula (I):

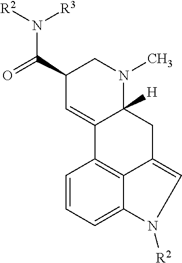

where exemplary values of the R groups in the above substituted chemical structure can be one or more of those represented in the following table:

| Name | R¹ | R² | R³ |
|---|---|---|---|
| LSD | H | CH₂CH₃ | CH₂CH₃ |
| Ergine | H | H | H |
| R-2-butyl | H | H | CH(CH₃)CH₂CH₃ |
| R-2-pentylamine | H | H | CH(CH₃)CH₂CH₂CH₃ |
| Analog of ergine | H | C₂H₅ | H |
| Analog of ergine | H | H | C₂H₅ |
| LSD | H | C₂H₅ | C₂H₅ |
| Analog of ergine | H | C₂H₅ | CH₂CH₂CH₃ |
| Analog of ergine | H | C₂H₅ | CH(CH₃)₂ |
| Analog of ergine | H | CH₂CH₂CH₃ | H |
| Analog of ergine | H | H | CH₂CH₂CH₃ |
| Analog of ergine | H | CH₂CH₂CH₃ | CH₂CH₂CH₃ |
| Analog of ergine | H | CH₂CH₂CH₃ | C₂H₅ |
| Analog of ergine | H | CH₂CH₂CH₃ | CH(CH₃)₂ |
| Analog of ergine | H | CH(CH₃)₂ | H |
| Analog of ergine | H | H | CH(CH₃)₂ |
| Analog of ergine | H | CH(CH₃)₂ | CH(CH₃)₂ |
| Analog of ergine | H | CH(CH₃)₂ | C₂H₅ |
| Analog of ergine | H | CH(CH₃)₂ | CH₂CH₂CH₃ |

In some embodiments, R¹ of formula (I) can be H, C₁-C₆-alkyl, OH, O—(C₁-C₆-alkyl), halogen, or C₁-C₄-haloalkyl; R² of formula (I) can be H, C₁-C₆-alkyl, OH, O—(C₁-C₆-alkyl), halogen, or C₁-C₄-haloalkyl; and R³ of formula (I) can be H, C₁-C₆-alkyl, OH, O—(C₁-C₆-alkyl), halogen, or C₁-C₄-haloalkyl.

In some embodiments, the 5-HT_{2A} agonist of the invention includes a compound having the following chemical formula (II):

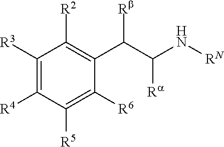

where exemplary values of the R groups in the above substituted chemical structure can be one or more of those represented in the following table:

| Name | R² | R³ | R⁴ | R⁵ | R^{α} | R^{β} |
|---|---|---|---|---|---|---|
| Mescaline | | OCH₃ | OCH₃ | OCH₃ | | |
| TMA | | OCH₃ | OCH₃ | OCH₃ | CH₃ | |
| TMA-2 | OCH₃ | | OCH₃ | OCH₃ | CH₃ | |
| methoxyDOB | OCH₃ | | Br | OCH₃ | CH₃ | OCH₃ |
| DOM | OCH₃ | | CH₃ | OCH₃ | | |
| DOB | OCH₃ | | Br | OCH₃ | | |
| DOI | OCH₃ | | I | OCH₃ | | |
| Sulfur analog | | OCH₃ | OCH₃ | SCH₃ | | |
| of mescaline | | | | | | |
| Sulfur analog | | OCH₃ | SCH₃ | OCH₃ | | |
| of mescaline | | | | | | |
| DOIB | OCH₃ | | CH₂CH(CH₃)₂ | OCH₃ | CH₃ | |
| DOTFM | OCH₃ | | CF₃ | OCH₃ | CH₃ | |

In some embodiments, R² of formula (II) can be OH, 0-(C₁-C₆-alkyl), —O—(C₂-C₆-alkyl)-N(R⁵)₂, or —O—(C₂-C₆-alkyl)-N(R^{x})₃+halogen-; R³ of formula (II) can be OH, 0-(C-C₆-alkyl), —O—(C₂-C₆-alkyl)-N(R^{X})₂, or —O—(C₂-C₆-alkyl)-N(R^{x})₃+halogen-; R⁴ of formula (II) can be halogen, C₁-C₂-haloalkyl, H, C₁-C₆-alkyl, C₁-C₆-alkyl sulfide, OH, O—(C₁-C₆-alkyl), —O—(C₂-C₆-alkyl)-N(R^{X})₂, or —O—(C₂-C₆-alkyl)-N(R^{X})₃+halogen-; R⁵ of formula (II) can be halogen, C₁-C₂-haloalkyl, H, C₁-C₆-alkyl, C₁-C₆-alkyl sulfide, OH, 0-(C₁-C₆-alkyl), —O—(C₂-C₆-alkyl)-N(R^{x})₂, or —O—(C₂-C₆-alkyl)-N(R^{X})₃+halogen-; R⁶ of formula (II) can be halogen, C₁-C₂-haloalkyl, H, C₁-C₆-alkyl, —S—(C₁-C₆-alkyl), OH, O—(C₁-C₆-alkyl), —O—(C₂-C₆-alkyl)-N(R⁵)₂, or —O—(C₂-C₆-alkyl)-N(R⁵)₃+halogen-; R^{p} is H, halogen, or C₁-C₆-alkyl; RR of formula (II) can be OH, 0-(C₁-C₆-alkyl), —O—(C₂-C₆-alkyl)-N(R⁵)₂, or —O—(C₂-C₆-alkyl)-N(R^{X})₃+halogen-; R^{N} of formula (II) can be halogen, C₁-C₂-haloalkyl, H, C₁-C₆-alkyl, C₁-C₆-alkyl sulfide, OH, 0-(C₁-C₆-alkyl), —O—(C₂-C₆-alkyl)-N(R^{X})₂, or —O—(C₂-C₆-alkyl)-N(R^{X})₃+halogen-; and R^{X} is independently H or C₁-C₄-alkyl.

In some embodiments, the 5-HT_{2A} agonist of the invention includes a compound having the following chemical formula (III):

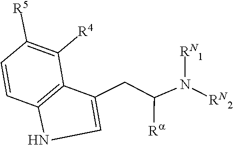

where exemplary values of the R groups in the above substituted chemical structure can be one or more of those represented in the following table:

| Name | R^{N}₁ | R^{N}₂ | R^{α} | R⁴ | R⁵ | R⁶ | R⁷ |
|---|---|---|---|---|---|---|---|
| 6-fluoro-psilocin | C | C | H | OH | H | F | H |
| 7-fluoro-psilocin | C | C | H | OH | H | H | F |
| 4-fluoro-5-methoxy- | C | C | H | F | OCH₃ | H | H |
| DMT | | | | | | | |
| 6-fluoro-5-methoxy- | C | C | H | H | OCH₃ | F | H |
| DMT | | | | | | | |
| α-Methyl-tryptamine | H | H | CH₃ | H | H | H | H |
| Serotonin | H | H | H | H | OH | H | H |

In some embodiments, R^{N}₁ of formula (III) can be H, C₁-C₆-alkyl, OH, 0-(C₁-C₆-alkyl), halogen, or C₁-C₄-haloalkyl; R^{N2} of formula (III) can be H, C₁-C₆-alkyl, OH, 0-(C₁-C₆-alkyl), halogen, or C₁-C₄-haloalkyl; R^{p} of formula (III) can be H, C₁-C₆-alkyl, OH, 0-(C₁-C₆-alkyl), halogen, or C₁-C₄-haloalkyl; R⁴ of formula (III) can be H, C₁-C₆-alkyl, OH, 0-(C₁-C₆-alkyl), halogen, or C₁-C₄-haloalkyl; R⁵ of formula (III) can be H, C₁-C₆-alkyl, OH, 0-(C₁-C₆-alkyl), halogen, or C₁-C₄-haloalkyl; R⁶ of formula (III) can be H, C₁-C₆-alkyl, OH, 0-(C₁-C₆-alkyl), halogen, or C₁-C₄-haloalkyl; and R⁷ of formula (III) can be H, C₁-C₆-alkyl, OH, O—(C₁-C₆-alkyl), halogen, or C₁-C₄-haloalkyl.

### Formulations

Formulations of psychedelic agents for oral use include tablets containing the psychedelic agent in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. For example, the coating may be adapted to release a psychedelic agent in a predetermined pattern (e.g., in order to achieve a controlled release formulation) or it may be adapted not to release the psychedelic agent until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the psychedelic agent). The coating may be applied on the solid dosage form in a similar manner as that described in Encyclopedia of Pharmaceutical Technology (eds. J. Swarbrick and J. C. Boylan, 1988-1999, Marcel Dekker, New York).

Formulations for oral use may also be presented as chewable tablets, or as hard gelatin capsules wherein the psychedelic agent is mixed with an inert solid diluent (e.g., potato starch, lactose, microcrystalline cellulose, calcium carbonate, calcium phosphate or kaolin), or as soft gelatin capsules wherein the psychedelic compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Powders, dispersible powders, or granules suitable for preparation of an aqueous suspension by addition of water are convenient dosage forms for oral administration of psychedelic agents. Formulation as a suspension provides the psychedelic agent in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Hayes (Remington: The Science and Practice of Pharmacy, volume I and volume 1l. Twenty-second edition. Philadelphia, 2012).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the psychedelic compound, the composition may include suitable parenterally acceptable carriers and/or excipients. The psychedelic agent may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the psychedelic agent is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### Example 1. Phase 1b Clinical Trial

With the emergence of psychoactives (e.g., psychedelics) as evidence-based psychiatric therapy, researchers have given special focus on optimization of the environment in which a participant undergoes this treatment. Participant perceptions are highly influenced by the setting of the study. Participants have heightened vulnerability, sensitivity, and suggestibility, while under the influence of a psychoactive drug. Context in which the therapy takes place is therefore important and has therapeutic value. Context also effects a participant's feeling of safety and trust. There is a growing concern of malpractice by those administering these drugs, as reports of sexual abuse are widespread.

Modern studies in human volunteers generally follow the comprehensive safety guidelines set forth in a Johnson et al, 2008 (Johnson, Richards, & Griffiths, Journal of Psychopharmacology 2008, 22(6): 603-620), which include the recommended presence of two attendants for each patient during a psychedelic treatment session. However, to viably integrate psychedelic treatments into standard psychiatric practice, new solutions are required to minimize staffing burdens associated with care delivery, ensure patient safety, enhance treatment efficacy, and improve attendant performance.

In this Phase 1 b trial, simultaneous dosing of participant triads was accomplished. This Phase 1, single-center, dose-escalation study used both open-label and double-blind placebo-controlled crossover design studies to evaluate the safety and tolerability of low doses of LSD ranging from 50 μg to 100 μg in healthy volunteers. A total of 32 healthy male or female subjects aged 21 to 65 years were enrolled.

### Monitoring/Safety Model

Remote monitoring of the sessions was conducted from a separate room by a clinically trained remote monitor to ensure clinical compliance. This allowed a single attendant to monitor patient treatment for the duration of acute psychoactive drug effects without specialized clinical certification. A psychiatrist was on-call and available but not attending (i.e., on premise). In addition to remotely monitoring the patient, the attendant was able to summon support staff (e.g., to facilitate bathroom breaks, temporary attendant coverage, clinical advice/questions, etc.) via a messaging on application. Remote monitoring further provided a means for subsequent training of attendants (e.g., through debriefing or training of future attendants) using recorded video and/or audio session data (e.g., processed or unprocessed).

### Safety Results

No serious adverse events were observed, and no participant discontinued due to an adverse event. Qualitative analysis of structured interviews revealed that all subjects reported feeling safe. 20 subjects expressed that having an attendant was important. Seven subjects said that an attendant was not essential for them to feel safe. Subjects were 100% compliant with all tasks, and 100% were retained in the trial.

### Video Results

Video and audio data were captured from the treatment rooms, aspects of which can be correlated with whether a situation required intervention from the attendant, e.g., based on questionnaires filled out by attendants about their subjects behavior throughout the day of dosing.

A key finding from this data is that movement inside the room was indicative of distress and anxiety in the subject. In four out of five videos of subjects who required intervention by the monitor, excessive movement throughout the room was observed by video data prior to intervention. In these four cases, the attendant rated the subject's anxiety and paranoid ideation as exceeding 25 on a 100-point scale.

Long conversation between an attendant and the subject that were inaudible to the monitor also resulted in intervention by the attendant due to deviation from clinical conduct guidelines.

Attendant ratings of unresponsiveness of a subject to the attendant's questions correlated with reports of strong anxiety (r=0.47). Additionally, reports of strong anxiety were correlated with paranoid ideation (r=0.41).

### Example 2. Escalation/Intervention

Phase 1b data revealed that video monitoring was capable of identifying the need for additional clinical support, though infrequent, and would thus enhance the cost-efficiency of psychoactive care delivery by increasing the efficiency of scarce clinical resources.

Specifically, methods of escalation include video monitoring (e.g., remote video monitoring) to identify when additional assistance or intervention is required in the context of psychoactive drug therapy, e.g., upon detection of excessive movement of the subject in the room (but not sudden motor movements). Additionally or alternatively, intervention is triggered by (1) lengthy inaudible conversation between subject and attendant and/or (2) expressions of paranoia or discomfort by the subject, e.g., as dialogue/spoken word, or as unresponsiveness to attendant queries.

Once an escalation criterion is satisfied, the remote monitor intervenes by (1) checking in with the attendant via secured messaging; (2) summoning back-up clinical staff to support the attendant; and/or (3) alerting the on-call psychiatrist.

### Example 3. Reducing the Risk of Clinical Misconduct

Rates of prevalence of sexual misconduct in such clinical settings range from 7-12% among general mental health practitioners in the United States (Celenza, 2007), encompassing approximately 2.5% of women and 9.4% of men (Pope, Keith-Spiegel, & Tabachnick, 1986; as cited in Koocher & Keith-Spiegel, 2008). Professionals who engage in this behavior are subject to ethical complaints, malpractice suits, and licensing board hearings (Pope et al., 1986), and deleterious outcomes for clients (Pope, 2001), as well as students, supervisees, and therapists (Koocher & Keith-Spiegel, 2008) are often extensive and multifaceted.

Methods and systems of the invention are employed as a means to reduce the risk of clinical misconduct in several ways. First, digital recording of session data provides a heightened sense of awareness and/or accountability to the attendant. Second, digital recording of session data allows for monitoring of interactions between attendant and patient and can alert a remote monitor of clinical misconduct that may be developing or developing, thereby enabling a means for intervention.

### Example 4. Using EEG to Assess a Patient's Readiness for Release after Completing Treatment

A 25-year old patient diagnosed with major depression has scored 40 on the 10-item Montgomery-Asberg Depression Rating Scale (MADRS) and is identified as a candidate for combination ketamine infusion therapy. While this treatment may be administered to alleviate symptoms of depression, the patient is monitored after treatment in the clinic for a period of time to ensure that residual drug effects have subsided prior to release.

Utilizing a dry-electrode EEG headset, resting state EEG data is collected at baseline, during the treatment, and directly after treatment. Alternatively, the EEG headset is placed on the patient's head only after the infusion is stopped.

The EEG data stream is used as input for an algorithm on a local device which determines the level of residual psychoactive effects of the drug by detecting, e.g., changes in alpha spectral power and/or spectral power changes in the gamma band and/or changes in a measure of spontaneous signal diversity. Alternatively, the algorithm may provide the output from a remote server.

Thirty minutes after the patient's infusion ends, the clinician is notified that the psychoactive drug effects of ketamine have subsided and that the patient may be released based upon an assessment of the EEG signal. Should the results of the automated analysis of EEG signal indicate that the patient is still under the effects of the drug, the patient will not be released from the clinic and will undergo further monitoring.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of monitoring a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; and
(ii) transferring the session data from the access module to a remote monitor.

2. The method of claim 1, further comprising:
(iii) within the access module, processing the session data to derive a patient response metric.

3. The method of claim 2, further comprising:
(iv) transferring the patient response metric from the access module to the remote monitor.

4. A method of monitoring a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; and
(ii) within the access module, processing the session data to derive a patient response metric indicative of a patient response to the psychoactive therapy session.

5. The method of claim 4, further comprising:
(iv) transferring unprocessed session data from the access module to the remote monitor; and/or
(iii) transferring the patient response metric from the access module to a remote monitor.

6. The method of any one of claims 1-3 or 5, wherein the session data is transferred from the access module to the remote monitor in real-time.

7. The method of any one of claims 1-6, wherein the session data comprises digitally recorded data.

8. The method of claim 7, wherein the digitally recorded data comprises video and/or audio data.

9. The method of claim 7 or 8, wherein the digitally recorded data comprises wearable sensor data.

10. The method of any one of claims 7-9, wherein the digitally recorded data comprises EEG sensor data or infrared sensor data.

11. The method of any one of claims 7-10, wherein the patient response metric comprises a measure of movement throughout the treatment setting derived from the digitally recorded data.

12. The method of claim 11, wherein a high measure of movement throughout the treatment setting indicates an adverse patient response to the psychoactive therapy session.

13. The method of any one of claims 7-12, wherein the patient response metric comprises a measure of conversation between the patient and the attendant.

14. The method of claim 13, wherein a lengthy inaudible conversation between the patient and the attendant indicates a deviation from protocol.

15. The method of claim 12 or 14, further comprising alerting a clinical support staff member of the adverse patient response.

16. The method of claim 15, wherein the clinical support staff member is the remote monitor, a non-certified staff member, a certified, clinician, or a physician.

17. The method of claim 15 or 16, wherein, after alerting the clinical support staff member of the adverse patient response, the clinical support staff member:
(a) intervenes in the psychoactive therapy session;
(b) alerts another clinical support staff member; and/or
(c) escalates the alert to a physician.

18. The method of claim 17, wherein part (b) further comprises escalating the alert to a physician.

19. The method of claim 17 or 18, wherein the physician intervenes in the psychoactive therapy session.

20. The method of claim 19, wherein the intervention is a remote intervention or a local intervention.

21. The method of any one of claims 1-20, wherein the session data comprises manually recorded data provided by the attendant.

22. The method of claim 21, wherein the manually recorded data comprises a rating of unresponsiveness.

23. The method of claim 21 or 22, wherein the manually recorded data comprises a patient self-report.

24. The method of claim 22 or 23, wherein the patient response metric comprises a measure of anxiety and/or paranoid ideation.

25. The method of claim 24, wherein a high measure of anxiety and/or paranoid ideation indicates an adverse patient response to the psychoactive therapy session.

26. The method of claim 25, further comprising alerting a clinical support staff member of the adverse patient response.

27. The method of claim 26, wherein the clinical support staff member is the remote monitor, a non-certified staff member, a certified, clinician, or a physician.

28. The method of claim 26 or 27, wherein, after alerting the clinical support staff member of the adverse patient response, the clinical support staff member:
(a) intervenes in the psychoactive therapy session;
(b) alerts another clinical support staff member; and/or
(c) escalates the alert to a physician.

29. The method of claim 28, wherein part (b) further comprises escalating the alert to a physician.

30. The method of claim 28 or 29, wherein the physician intervenes in the psychoactive therapy session.

31. The method of claim 30, wherein the intervention is a remote intervention or a local intervention.

32. A method of intervening in a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting;
(ii) transferring the session data from the access module to a remote monitor;
(iii) identifying an aberrance in the session data; and
(iv) summoning clinical support to the treatment setting, thereby intervening in the psychoactive therapy session.

33. The method of claim 32, wherein the aberrance is misconduct by the attendant.

34. The method of claim 32 or 33, further comprising:
(v) within the access module, processing the session data to derive a patient response metric.

35. The method of claim 34, further comprising:
(iv) transferring the patient response metric from the access module to the remote monitor.

36. The method of any one of claims 32-35, wherein the aberrance is an adverse patient response to the psychoactive therapy session.

37. The method of any one of claims 1-36, wherein the access module and/or remote monitor receives session data from multiple treatment settings simultaneously.

38. The method of any one of claims 1-37, wherein the remote monitor is not a physician.

39. The method of claim 38, wherein the remote monitor is equipped with a secure connection to a physician.

40. A method of intervening in a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting;
(ii) within the access module, processing the session data to derive a patient response metric indicative of a patient response to the psychoactive therapy session;
(iii) identifying an aberrance based on the patient response metric; and
(iv) summoning a clinical support staff member to the treatment setting, thereby intervening in the psychoactive therapy session.

41. The method of claim 40, wherein the clinical support staff member is the remote monitor, a non-certified staff member, a certified, clinician, or a physician.

42. The method of claim 40 or 41, wherein, after alerting the clinical support staff member of the adverse patient response, the clinical support staff member:
(a) intervenes in the psychoactive therapy session;
(b) alerts another clinical support staff member; and/or
(c) escalates the alert to a physician.

43. The method of claim 42, wherein part (b) further comprises escalating the alert to a physician.

44. The method of claim 42 or 43, wherein the physician intervenes in the psychoactive therapy session.

45. The method of claim 44, wherein the intervention is a remote intervention or a local intervention.

46. The methods of any one of claims 40-45, further comprising transferring the patient response metric from the access module to a remote monitor.

47. The method of any one of claims 40-46, further comprising transferring unprocessed session data from the access module to the remote monitor.

48. The method of any one of claims 32-47, wherein the session data comprises digitally recorded data.

49. The method of claim 48, wherein the digitally recorded data comprises video and/or audio data.

50. The method of claim 48 or 49, wherein the digitally recorded data comprises wearable sensor data.

51. The method of any one of claims 48-50, wherein the digitally recorded data comprises EEG sensor data or infrared sensor data.

52. The method of any one of claims 48-51, wherein the patient response metric comprises a measure of movement throughout the treatment setting derived from the digitally recorded data.

53. The method of claim 52, wherein the aberrance is an adverse patient response to the psychoactive therapy session indicated by a high measure of movement throughout the treatment setting.

54. The method of any one of claims 48-53, wherein the patient response metric comprises a measure of conversation between the patient and the attendant.

55. The method of claim 54, wherein the aberrance is a deviation from protocol indicated by an inaudible conversation between the patient and the attendant.

56. The method of any one of claims 32-55, wherein the session data comprises manually recorded data provided by the attendant.

57. The method of claim 56, wherein the manually recorded data comprises a rating of unresponsiveness.

58. The method of claim 56 or 57, wherein the manually recorded data comprises a patient self-report.

59. The method of any one of claims 56-58, wherein the patient response metric comprises a measure of anxiety and/or paranoid ideation.

60. The method of claim 59, wherein the aberrance is an adverse patient response to the psychoactive therapy session indicated by a high measure of anxiety and/or paranoid ideation.

61. A method of training clinical staff for monitoring a psychoactive therapy session, the method comprising:
(i) performing a method of any one of claims 1-60;
(ii) providing one or more trainees;
(iii) presenting session data to the one or more trainees; and
(iv) providing a characterization of the session data to the one or more trainees, thereby training clinical staff.

62. The method of claim 61, wherein the clinical staff is an attendant, a remote monitor, a clinical professional, or a physician.

63. A method of conducting a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting;
(ii) within the access module:
(a) processing the session data to derive a first patient response metric indicative of a psychoactive drug effect: and
(b) processing the session data to derive a subsequent patient response metric indicative of the psychoactive drug effect; and
(iii) comparing the first patient response metric with the subsequent patient response metric; and
(iv) based on the comparison of (iii), determining whether to dismiss the patient from the treatment setting.

64. The method of claim 63, wherein the access module comprises a processor, and wherein the processor performs the comparison of (iii).

65. A method of editing session data from a psychoactive therapy session, the method comprising:
(i) receiving session data via an access module, wherein the session data is transmitted to the access module from the psychoactive therapy session; and
(ii) within the access module:
(a) processing the session data comprising a pertinent dataset and an extraneous dataset to derive a patient response metric indicative of a patient response to the psychoactive therapy session, wherein the patient response metric is preferentially based on the pertinent dataset; and
(b) producing an edited dataset by isolating the pertinent dataset from the extraneous dataset.

66. A method of generating a patient response database, the method comprising:
(i) via an access module, receiving session data from a first psychoactive therapy session and session data from a second psychoactive therapy session, wherein the first and second psychoactive therapy sessions are each conducted in a treatment setting comprising a patient and an attendant, wherein no physician is present in the treatment setting during either of the first and second psychoactive therapy sessions;
(ii) within the access module: (a) processing the session data from the first psychoactive therapy session to derive a first patient response metric indicative of a first patient response to the psychoactive therapy session; and (b) processing the session data from the second psychoactive therapy session to derive a second patient response metric indicative of a second patient response to the psychoactive therapy session;
(iii) recording the first patient response metric and the second patient response metric to a computer-readable medium, thereby generating a patient response database.

67. The method of claim 66, further comprising deriving an average patient response from the first patient response and the second patient response.

68. A method of monitoring a psychoactive therapy session, the method comprising:
(i) receiving session data via an access module, wherein the session data is transmitted to the access module from the psychoactive therapy session, wherein the psychoactive therapy session is conducted in a treatment setting comprising a patient and an attendant, and wherein no physician is present in the treatment setting; and
(ii) within the access module, processing the session data to derive a patient response metric indicative of a patient response to the psychoactive therapy session, wherein the processing draws reference data from a patient response database generated by the method of claim 66 or 67.

69. The method of any one of claims 1-68, wherein the psychoactive therapy session is for treatment of a psychological condition.

70. The method of claim 69, wherein the psychological condition is depression, anxiety, PTSD, or substance abuse, alcoholism, anorexia nervosa, bulimia, obesity, eating disorders, or obsessive-compulsive disorder.

71. The method of any one of claims 1-68, wherein the psychoactive therapy session is for treatment of a non-psychological condition.

72. The method of claim 71, wherein the non-psychological condition is an autoimmune condition or a pain.

73. The method of any one of claims 1-68, wherein the psychoactive therapy session is part of a neurorehabilitation program.

74. The method of any one of claims 1-68, wherein the psychoactive therapy session is for enhancing well-being.

75. The method of any one of claims 1-70, wherein the psychoactive therapy session is conducted in the presence of one and only one attendant.

76. The method of any one of claims 1-75, wherein the patient and the attendant are the only people in the treatment setting during the psychoactive therapy session.

77. The method of any one of claims 1-76, wherein the psychedelic therapy session comprises a perceptual dose of a psychoactive agent.

78. The method of any one of claims 1-77, wherein the psychoactive therapy session comprises administration of an agent selected from the group consisting of a 5-HT_{2A} receptor agonist, an empathogenic agent, and a dissociative agent.

79. The method of claim 78, wherein the psychoactive therapy session comprises administration of a 5-HT_{2A} receptor agonist.

80. The method of claim 79, wherein the 5-HT_{2A} receptor agonist is selected from lysergic acid diethylamide (LSD), psilocybin, DOI (±)-1-(2,5-dimethoxyphenyl)-2-aminopropane hydrochloride; (R)-DOI ((R)-1-(2,5-dimethoxy-4-iodophenyl)-2-aminopropane); LA-SS-Az (2'S,4'S)-(+)-9,10-Didehydro-6-methylergoline-8β-(trans-2,4-dimethylazetidide); 2C-BCB (4-Bromo-3,6-dimethoxybenzocyclobuten-1-yl)methylamine) ayahuasca; 3,4,5-trimethoxyphenethylamine (mescaline); 5-methoxy-N,N-dimethyltryptamine (5-meo-DMT); ibogaine; voacangine; noribogaine; coronaridine; 18-methoxycoronaridine (18-mc); methoxyethyl 18-MC; 18-methylaminocoronaridine (18-MAC); 2-methoxyethyl-18-methoxycoronaridinate; a compound of 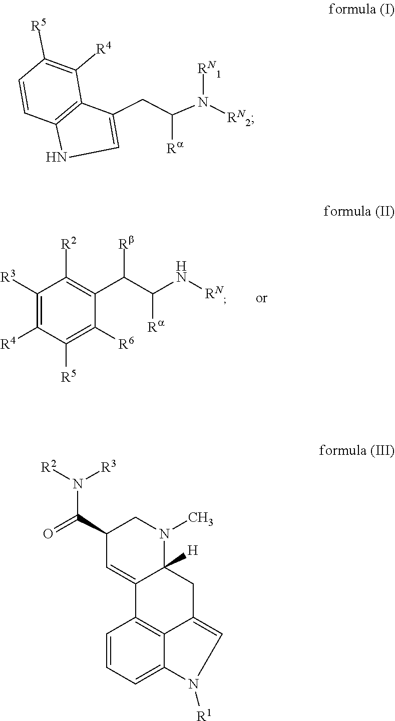
or a pharmaceutically acceptable salt thereof.

81. The method of claim 78, wherein the psychoactive therapy session comprises administration of an empathogenic agent.

82. The method of claim 81, wherein the empathogenic agent is MDMA.

83. The method of claim 78, wherein the psychoactive therapy session comprises administration of a dissociative agent.

84. The method of claim 83, wherein the dissociative agent is ketamine, esketamine, or arketamine.

85. A system comprising an access module configured to perform the method of any one of claims 1-84.

86. The method of any one of claims 61-85, wherein the session data comprises wearable sensor data.

87. The method of claim 86, wherein the sensor data comprises EEG sensor data or infrared sensor data.
